(19) United States
(12) Patent Application Publication
(10) Pub. No.: US 2025/0051246 A1
(43) Pub. Date: Feb. 13, 2025

(54) Deuterated Antiviral Compounds

(71) Applicant: Pfizer Inc., New York, NY (US)

(72) Inventors: Karen Jean COFFMAN, Pawcatuck, CT (US); Dafydd Rhys OWEN, Concord, MA (US); Matthew Richard REESE, Mystic, CT (US); Matthew Forrest SAMMONS, Quincy, MA (US); Raman SHARMA, Groton, CT (US); Jamison Bryce TUTTLE, Marblehead, MA (US); Qingyi YANG, Lexington, MA (US)

(73) Assignee: Pfizer Inc., New York, NY (US)

(21) Appl. No.: 18/799,069

(22) Filed: Aug. 9, 2024

Related U.S. Application Data
(60) Provisional application No. 63/519,164, filed on Aug. 11, 2023.

(51) Int. Cl. C07B 59/00; A61K 31/4025; A61P 31/14; C07D 403/12

(52) U.S. Cl. CPC C07B 59/004; A61K 31/4025; A61P 31/14; C07D 403/12; C07B 2200/05

(57) ABSTRACT

The invention relates to compounds of Formula (I)

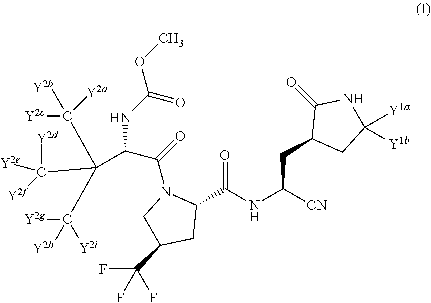

wherein Y^{1a}, Y^{1b}, Y^{2a}, Y^{2b}, Y^{2c}, Y^{2d}, Y^{2e}, Y^{2f}, Y^{2g}, Y^{2h} and Y²ⁱ are as defined herein and to their use in medicine; to compositions containing them; to processes for their preparation; and to intermediates used in such processes. The compounds of Formula (I) may be useful in the treatment of coronavirus infections and coronavirus infection related disorders.

### BACKGROUND OF THE INVENTION

The present invention relates to novel deuterated antiviral compounds. The invention also relates to the preparation of the compounds and intermediates used in the preparation, compositions containing the compounds, and uses of the compounds including as 3CL protease inhibitors which are useful for the treatment of coronavirus diseases such as COVID-19.

Many current medicines suffer from poor absorption, distribution, metabolism and/or excretion (ADME) properties that prevent their wider use or limit their use in certain indications. Poor ADME properties are also a major reason for the failure of drug candidates in clinical trials. While formulation technologies and prodrug strategies can be employed in some cases to improve certain ADME properties, these approaches often fail to address the underlying ADME problems that exist for many drugs and drug candidates. One such problem is rapid metabolism that causes a number of drugs, which otherwise would be highly effective in treating a disease, to be cleared too rapidly from the body. A possible solution to rapid drug clearance is frequent or high dosing to attain a sufficiently high plasma level of drug. This, however, introduces a number of potential treatment problems such as poor patient compliance with the dosing regimen, side effects that become more acute with higher doses, and increased cost of treatment. A rapidly metabolized drug may also expose patients to undesirable toxic or reactive metabolites.

Another ADME limitation that affects many medicines is the formation of toxic or biologically reactive metabolites. As a result, some patients receiving the drug may experience toxicities, or the safe dosing of such drugs may be limited such that patients receive a suboptimal amount of the active agent. In certain cases, modifying dosing intervals or formulation approaches can help to reduce clinical adverse effects, but often the formation of such undesirable metabolites is intrinsic to the metabolism of the compound.

Hydrogen is the most abundant element in the universe and has three isotopes that exist naturally: protium (1H, hereinafter abbreviated as H), which has one proton and one electron, with a 99.9844% abundance, deuterium (2H, hereinafter abbreviated as D), bearing an added neutron, with a 0.0156% abundance and tritium (3H, hereinafter abbreviated as T), bearing two added neutrons, and which is present in traces.

A potentially attractive strategy for improving a drug's metabolic properties is deuterium modification. In this approach, one attempts to slow the CYP-mediated metabolism of a drug or to reduce the formation of undesirable metabolites by replacing one or more hydrogen atoms with deuterium atoms. Deuterium is a safe, stable, non-radioactive isotope of hydrogen. Compared to hydrogen, deuterium forms stronger bonds with carbon. In select cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a small percentage of approved drugs (see, e.g., Blake, M I et al, J Pharm Sci, 1975, 64:367-91; Foster, AB, Adv Drug Res 1985, 14:1-40 (“Foster”); Kushner, D J et al, Can J Physiol Pharmacol 1999, 79-88; Fisher, M B et al, Curr Opin Drug Discov Devel, 2006, 9:101-09 (“Fisher”)). The results have been variable and unpredictable. For some compounds deuteration caused decreased metabolic clearance in vivo. For others, there was no change in metabolism. Still others demonstrated increased metabolic clearance. The variability in deuterium effects indicates that there is inherent uncertainty when using deuterium modification as a drug design strategy for inhibiting adverse metabolism (see Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of metabolism will differ from that of its non-deuterated counterpart. See, for example, Fukuto et al. (J. Med. Chem. 1991, 34, 2871-76). Many drugs have multiple sites where metabolism is possible. The site(s) where deuterium substitution is required and the extent of deuteration necessary to see an effect on metabolism, if any, will be different for each drug.

The impact of deuteration of a compound on absorption, distribution, metabolism, excretion and toxicity (ADMET) parameters goes beyond metabolism (see Di Martino, R. M. C., Maxwell, B. D. and Pirali, T.; Nature Reviews Drug Discovery https://doi.org/10.1038/s41573-023-00703.8; published Jun. 5, 2023. Although the difference in lipophilicity between H and D is negligible, when multiple D atoms are present the additive effect could lead to significant differences in terms of plasma protein binding, extent and/or rate of absorption and P-glycoprotein efflux, all effects that warrant further investigation. A reduction in lipophilicity due to the introduction of multiple D atoms could be sufficient to reduce the passage of deuterated molecules across the blood-brain barrier (see Wang, X.-M. et al. Effect of deuteration on the single dose pharmacokinetic properties and postoperative analgesic activity of methadone. Drug. Metab. Pharmacokinet. 47, 100477 (2022)). Other potential benefits of deuteration that are emerging but are still not completely understood include a potential effect of D on both drug target interactions and time-dependent inhibition of CYP enzymes (see for example Sun, L.-Q. et al. Discovery of BMS-986144, a third-generation, pan-genotype NS3/4A protease inhibitor for the treatment of hepatitis C virus infection. J. Med. Chem. 63, 14740-14760 (2020); Spock, M. et al. Discovery of VU6028418: a highly selective and orally bioavailable M4 muscarinic acetylcholine receptor antagonist. ACS Med. Chem. Lett. 12, 1342-1349 (2021); Xu, Z. et al. Evaluation of efficacy and safety after replacement of methyl hydrogen with deuterium at methyl formate of clopidogrel. Eur. J. Pharm. Sci. 172, 106157 (2022); and Shvartsbart, A. et al. Discovery of potent and selective inhibitors of wild-type and gatekeeper mutant fibroblast growth factor receptor (FGFR) 2/3. J. Med. Chem. 65, 15433-15442 (2022). Inhibition of cytochromes can be undesirable for a drug product as it might lead to drug-drug interactions in patients undergoing multiple therapies. Deuterium incorporation might also reduce the time-dependent inhibition of CYP enzymes, although the reason behind this effect is unclear.

The present compounds of Formula (I) are deuterated analogs of N-(methoxycarbonyl)-3-methyl-L-valyl-(4R)—N-{(1S)-1-cyano-2-[(3S)-2-oxopyrrolidin-3-yl]ethyl}-4-(trifluoromethyl)-L-prolinamide which is described in Example 95 of PCT International Application Publication WO 2021/250648 published Dec. 16, 2021 and U.S. Pat. No. 11,351,149 which issued Jun. 7, 2022. In studies, including human clinical studies, it has been observed that N-(methoxycarbonyl)-3-methyl-L-valyl-(4R)—N-{(1S)-1-cyano-2-[(3S)-2-oxopyrrolidin-3-yl]ethyl}-4-(trifluoromethyl)-L-prolinamide can undergo oxidative metabolism. Accordingly, there remains a need for improved antiviral 3CL protease inhibitor compounds. The compounds, combinations and methods of the present invention are believed to have one or more advantages, such as enhanced metabolic stability and improved pharmacokinetic and pharmacodynamic properties.

### SUMMARY OF THE INVENTION

The present invention provides, in part, compounds of Formula (I). Such compounds may inhibit the activity of the coronavirus 3CL protease and may be useful in the treatment, prevention, suppression of coronavirus infections, such as COVID-19, or coronavirus associated diseases or disorders such as long COVID. Also provided are pharmaceutical compositions, comprising the compounds of the invention, alone or in combination with additional antiviral therapeutic agents. The present invention also provides, in part, methods for preparing such compounds and compositions of the invention, and methods of using the foregoing. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

According to an embodiment of the invention there is provided a compound of Formula (I)

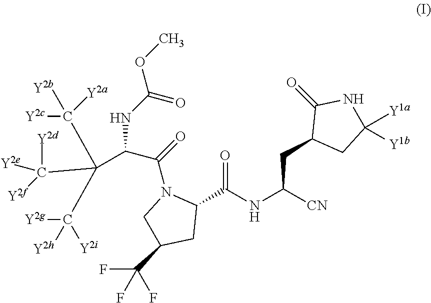

wherein
Y^{1a}, Y^{1b}, Y^{2a}, Y^{2b}, Y^{2c}, Y^{2d}, Y^{2e}, Y^{2f}, Y^{2g}, Y^{2h} and Y²ⁱ are each independently H or D; provided that at least one of Y^{1a}, Y^{1b}, Y^{2a}, Y^{2b}, Y^{2c}, Y^{2d}, Y^{2e}, Y^{2f}, Y^{2g}, Y^{2h} and Y²ⁱ is D.

Described below are embodiments of the invention, where for convenience Embodiment 1 (E1) is identical to the embodiment of Formula (I) provided above.

It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of the embodiments of the invention and the Examples included herein. It is to be understood that this invention is not limited to specific synthetic methods of making that may of course vary. It is to be also understood that the terminology used herein is for the purpose of describing specific embodiments only and is not intended to be limiting.

E1 A compound of Formula (I) as defined above.

E2 A compound of embodiment E1 wherein Y^{1a} and Y^{1b} are D; and Y^{2a}, Y^{2b}, Y^{2c}, Y^{2d}, Y^{2e}, Y^{2f}, Y^{2g}, Y^{2h} and Y²ⁱ are H.

E3 A compound of embodiment E1 wherein Y^{1a} is D; and Y^{1b}, Y^{2a}, Y^{2b}, Y^{2c}, Y^{2d}, Y^{2e}, Y^{2f}, Y^{2g}, Y^{2h} and Y²ⁱ are H.

E4 A compound of embodiment E1 wherein Y^{2a} is D; and Y^{1a}, Y^{1b}, Y^{2b}, Y^{2c}, Y^{2d}, Y^{2e}, Y^{2f}, Y^{2g}, Y^{2h} and Y²ⁱ are H.

E5 A compound of any one of embodiments E1 to E3 wherein Y^{2a} is D; and Y^{2b}, Y^{2c}, Y^{2d}, Y^{2e}, Y^{2f}, Y^{2g}, Y^{2h} and Y²ⁱ are H.

E6 A compound of any one of embodiments E1 to E3 wherein Y^{2a} and Y^{2b} are D; and Y^{2c}, Y^{2d}, Y^{2e}, Y^{2f}, Y^{2g}, Y^{2h} and Y²ⁱ are H.

E7 A compound of any one of embodiments E1 to E3 wherein Y^{2a}, Y^{2b} and Y^{2c} are D; and Y^{2d}, Y^{2e}, Y^{2f}, Y^{2g}, Y^{2h} and Y²ⁱ are H.

E8 A compound of any one of embodiments E1 to E3 wherein Y^{2a}, Y^{2b}, Y^{2c} and Y^{2d} are D; and Y^{2e}, Y^{2f}, Y^{2g}, Y^{2h} and Y²ⁱ are H.

E9 A compound of any one of embodiments E1 to E3 wherein Y^{2a}, Y^{2b}, Y^{2c}, Y^{2d} and Y^{2e} are D; and Y^{2f}, Y^{2g}, Y^{2h} and Y²ⁱ are H.

E10 A compound of any one of embodiments E1 to E3 wherein Y^{2a}, Y^{2b}, Y^{2c}, Y^{2d}, Y^{2e} and Y^{2f} are D; and Y^{2g}, Y^{2h} and Y²ⁱ are H.

E11 A compound of any one of embodiments E1 to E3 wherein Y^{2a}, Y^{2b}, Y^{2c}, Y^{2d}, Y^{2e}, Y^{2f} and Y^{2g} are D; and Y^{2h} and Y²ⁱ are H.

E12 A compound of any one of embodiments E1 to E3 wherein Y^{2a}, Y^{2b}, Y^{2c}, Y^{2d}, Y^{2e}, Y^{2f}, Y^{2g} and Y^{2h} are D; and Y²ⁱ is H.

E13 A compound of any one of embodiments E1 to E3 wherein Y^{2a}, Y^{2b}, Y^{2c}, Y^{2d}, Y^{2e}, Y^{2f}, Y^{2g}, Y^{2h} and Y²ⁱ are D.

E14 A compound of embodiment E1 wherein Y^{1a} and Y^{1b} are H; and Y^{2a}, Y^{2b}, Y^{2c}, Y^{2d}, Y^{2e}, Y^{2f}, Y^{2g}, Y^{2h} and Y²ⁱ are D.

E15 A compound of embodiment E1 wherein Y^{1a} and Y^{1b} are H; and any 2 to 7 of Y^{2a}, Y^{2b}, Y^{2c}, Y^{2d}, Y^{2e}, Y^{2f}, Y^{2g}, Y^{2h} and Y²ⁱ are D.

E16 A compound of embodiment E1 wherein Y^{1a} is D and Y^{1b} is H; and any 2 to 7 of Y^{2a}, Y^{2b}, Y^{2c}, Y^{2d}, Y^{2e}, Y^{2f}, Y^{2g}, Y^{2h} and Y²ⁱ are D.

E17 A compound of embodiment E1 wherein Y^{1a} and Y^{1b} are D; and any 2 to 7 of Y^{2a}, Y^{2b}, Y^{2c}, Y^{2d}, Y^{2e}, Y^{2f}, Y^{2g}, Y^{2h} and Y²ⁱ are D.

E18 A compound of embodiment E1 having Formula (Ia)

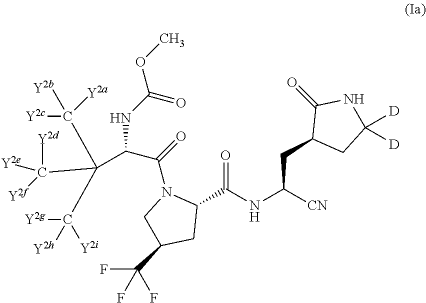

wherein 0 to 9 of Y^{2a}, Y^{2b}, Y^{2c}, Y^{2d}, Y^{2e}, Y^{2f}, Y^{2g}, Y^{2h} and Y²ⁱ are D.

E19 A compound of embodiment E1 having Formula (Ib)

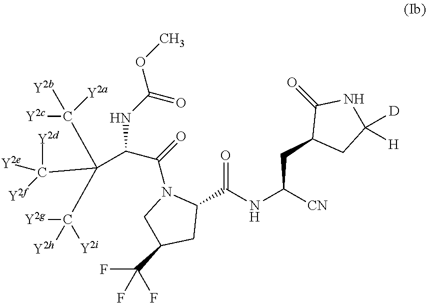

wherein 0 to 9 of Y^{2a}, Y^{2b}, Y^{2c}, Y^{2d}, Y^{2e}, Y^{2f}, Y^{2g}, Y^{2h} and Y²ⁱ are D.

E20 A compound of embodiment E1 having Formula (Ic)

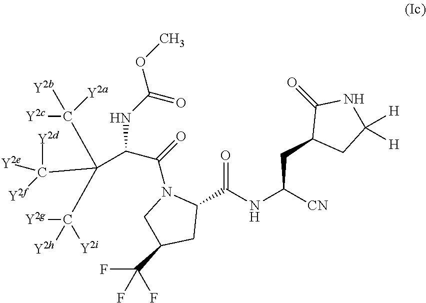

wherein 1 to 9 of Y^{2a}, Y^{2b}, Y^{2c}, Y^{2d}, Y^{2e}, Y^{2f}, Y^{2g}, Y^{2h} and Y²ⁱ are D.

E21 A compound of embodiment E1 having Formula (Id)

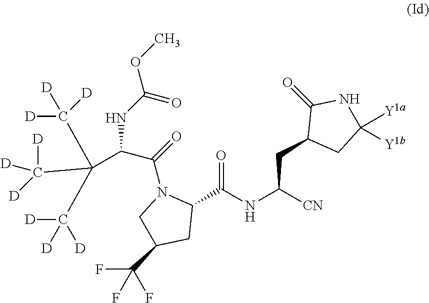

wherein Y^{1a} and Y^{1b} are each independently H or D.

E22 A compound of embodiment E1 having the Formula

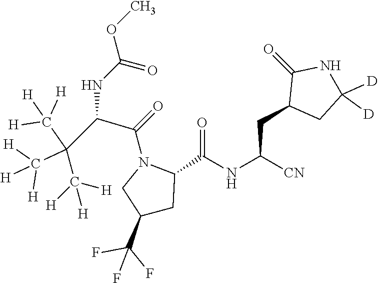 [SMILES: [2H]C1([2H])C[C@@H](C[C@@H](C#N)NC(=O)[C@@H]2C[C@@H](C(F)(F)F)CN2C(=O)[C@@H](NC(=O)OC)C(C)(C)C)C(=O)N1]

and is named N-(methoxycarbonyl)-3-methyl-L-valyl-(4R)—N-{(1S)-1-cyano-2-[(3S)-2-oxo(5,5-²H₂)pyrrolidin-3-yl]ethyl}-4-(trifluoromethyl)-L-prolinamide.

E23 A compound of embodiment E1 having the Formula

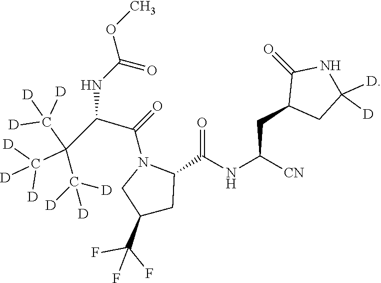

E24 A compound of embodiment E1 having the Formula

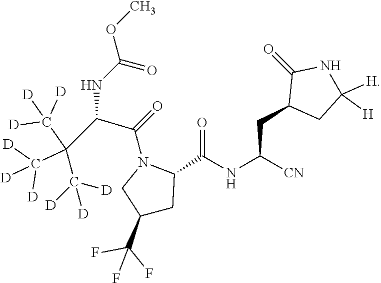

E25 A compound of embodiment E1 having the Formula

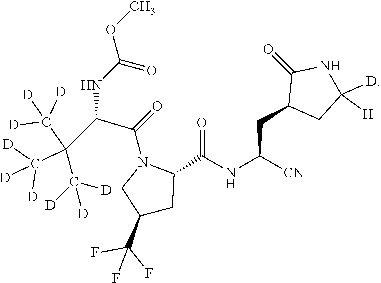

Any of the compounds described in embodiments E22 to E25 may be claimed individually or grouped together with one or more other compounds of embodiments E1 to E21.

E26 A pharmaceutical composition comprising a compound of any one of embodiments E1 to E25 and at least one pharmaceutically acceptable excipient.

E27 A method for treating a coronavirus infection or coronavirus infection related disorder, comprising administering to a subject in need thereof a therapeutically effective amount of a compound of any one of embodiments E1 to E25.

E28 The method of embodiment E27 wherein the coronavirus infection or coronavirus infection related disorder is selected from the group consisting of SARS-CoV-2 (COVID-19), SARS-CoV, MERS-Cov, HCoV-229E, HCoV-NL63, HCoV-OC43 and HCoV-HKU1 infections and the COVID-19 infection related disorder is long COVID.

E29 The method of embodiment E28 wherein the coronavirus infection is a SARS-CoV-2 (COVID-19) infection.

E30 A method for treating a coronavirus infection or coronavirus infection related disorder, comprising administering to a subject in need thereof a therapeutically effective amount of a compound of any one of embodiments E1 to E25 as a single agent.

E31 A method for treating coronavirus infection or coronavirus infection related disorder, comprising administering to a subject in need thereof a therapeutically effective amount of a compound of any one of embodiments E1 to E25, and further comprising administering a therapeutically effective amount of an additional antiviral therapeutic agent.

E32 A compound of any one of embodiments E1 to E25, for use as a medicament.

E33 A compound of any one embodiments E1 to E25, for use in the treatment of a coronavirus infection or coronavirus infection related disorder.

E34 The compound of embodiment E33 wherein the coronavirus infection or coronavirus infection related disorder is selected from the group consisting of SARS-CoV-2 (COVID-19), SARS-CoV, MERS-Cov, HCoV-229E, HCoV-NL63, HCoV-OC43 and HCoV-HKU1 infections and the COVID-19 infection related disorder is long COVID.

E35 The compound of embodiment 34 wherein the coronavirus infection is a SARS-CoV-2 (COVID-19) infection.

E36 Use of a compound of any one of embodiments E1 to E25 for the manufacture of a medicament for the treatment of coronavirus infection or coronavirus infection related disorder is selected from the group consisting of SARS-CoV-2 (COVID-19), SARS-CoV, MERS-Cov, HCoV-229E, HCoV-NL63, HCoV-OC43 and HCoV-HKU1 infections and the COVID-19 infection related disorder is long COVID.

E37 The use of embodiment 36 wherein the coronavirus infection is a SARS-CoV-2 (COVID-19) infection.

E38 The compound of any one of E1 to E25 wherein each D has a deuterium enrichment factor of at least 1000 (15% deuterium incorporation), at least 2000 (30% deuterium incorporation), at least 3000 (45% deuterium incorporation), at least 3500 (52.5% deuterium incorporation), at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

E39 The compound of E38 wherein each D has a deuterium enrichment factor of at least 5000 (75% deuterium incorporation).

E40 The compound of E39 wherein each D has a deuterium enrichment factor of at least 6000 (90% deuterium incorporation).

E41 The compound of E40 wherein each D has a deuterium enrichment factor of at least 6333.3 (95% deuterium incorporation).

E42 The compound of E41 wherein each D has a deuterium enrichment factor of at least 6466.7 (97% deuterium incorporation).

E43 The compound of E42 wherein each D has a deuterium enrichment factor of at least 6600 (99% deuterium incorporation).

E44 The compound of E43 wherein each D has a deuterium enrichment factor of at least 6633.3 (99.5% deuterium incorporation).

E45 The compound of E22 wherein each D has a deuterium enrichment factor of at least 5000 (75% deuterium incorporation).

E46 The compound of E45 wherein each D has a deuterium enrichment factor of at least 6000 (90% deuterium incorporation).

E47 The compound of E46 wherein each D has a deuterium enrichment factor of at least 6333.3 (95% deuterium incorporation).

E48 The compound of E47 wherein each D has a deuterium enrichment factor of at least 6466.7 (97% deuterium incorporation).

E49 The compound of E48 wherein each D has a deuterium enrichment factor of at least 6600 (99% deuterium incorporation).

E50 The compound of E49 wherein each D has a deuterium enrichment factor of at least 6633.3 (99.5% deuterium incorporation).

Each of the embodiments described herein may be combined with any other embodiment(s) described herein not inconsistent with the embodiment(s) with which it is combined. In addition, any of the compounds described in the Examples, may be claimed individually or grouped together with one or more other compounds of the Examples for any of the embodiment(s) described herein.

### Definitions

Unless otherwise defined herein, scientific and technical terms used in connection with the present invention have the meanings that are commonly understood by those of ordinary skill in the art.

The invention described herein suitably may be practiced in the absence of any element(s) not specifically disclosed herein.

“Compounds of the invention” include compounds of Formula I and the novel intermediates used in the preparation thereof. One of ordinary skill in the art will appreciate that compounds of the invention include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that compounds of the invention include solvates, hydrates, isomorphs, polymorphs and isotopically labelled versions thereof (including additional deuterium substitutions), where they may be formed.

As used herein, the singular form “a”, “an”, and “the” include plural references unless indicated otherwise. For example, “a” substituent includes one or more substituents.

As used herein, the term “about” when used to modify a numerically defined parameter (e.g., the dose of about 100, 200, 300, 400, 500 mg) means that the parameter may vary by as much as 10% below or above the stated numerical value for that parameter. For example, a dose of about 100 mg means 100 mg±10%, (i.e., it may vary between 90 mg and 110 mg), a dose of about 200 mg means 200 mg±10%, (i.e., it may vary between 180 mg and 220 mg), a dose of about 300 mg means 300 mg±10%, (i.e., it may vary between 270 mg and 330 mg), a dose of about 400 mg means 400 mg±10%, (i.e., it may vary between 360 mg and 440 mg) and a dose of about 500 mg means 500 mg±10%, (i.e., it may vary between 450 mg and 550 mg).

If substituents are described as being “independently selected” from a group, each substituent is selected independent of the other. Each substituent therefore may be identical to or different from the other substituent(s).

The term “pharmaceutically acceptable” means the substance (e.g., the compounds described herein) and any salt thereof, or composition containing the substance or salt of the invention is suitable for administration to a subject or patient.

A “pharmaceutical composition” refers to a mixture of one or more of the compounds of the invention, or a pharmaceutically acceptable salt, solvate, hydrate or prodrug thereof as an active ingredient, and at least one pharmaceutically acceptable excipient.

“Deuterium enrichment factor” as used herein means the ratio between the deuterium abundance and the natural abundance of deuterium, each relative to hydrogen abundance. An atomic position designated as having deuterium (i.e. “D”) typically has a deuterium enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

“Excipient” as used herein describes any ingredient other than the compound(s) of the invention. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugar, sodium chloride, or polyalcohol such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The term “treating”, “treat” or “treatment” as used herein embraces both preventative, i.e., prophylactic, and palliative treatment, i.e., relieve, alleviate, or slow the progression of the patient's disease (or condition), such as a coronavirus infection including COVID-19, or any sequellae associated with the disease such as long COVID.

As used herein, the term, “subject, “individual” or “patient,” used interchangeably, refers to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting (or slowing) further development of the pathology or symptomatology or both); and
(3) ameliorating the disease; for example, ameliorating a disease, condition or disorder in an individual that is experiencing or displaying the pathology or symptomatology of the disease, condition or disorder (i.e., reversing the pathology or symptomatology or both).

### Salts

Salts may include other salts of such compounds which are not necessarily pharmaceutically acceptable salts, which may be useful as intermediates for preparing compounds of Formula I. The compounds of Formula I of the present invention are not known to form salts but various intermediates used in the preparation of the compounds of Formula I are known to form salts.

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinofoate salts.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

Pharmaceutically acceptable salts of compounds of the invention (in this case intermediate compounds used to prepare the compounds of Formula I) may be prepared by methods well known to one skilled in the art, including but not limited to the following procedures
(i) by reacting a compound of the invention with the desired acid or base;
(ii) by removing an acid- or base-labile protecting group from a suitable precursor of a compound of the invention or by ring-opening a suitable cyclic precursor, for example, a lactone or lactam, using the desired acid or base; or
(iii) by converting one salt of a compound of the invention to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

### Solvates

The compounds of the invention, and pharmaceutically acceptable salts thereof, may exist in unsolvated and solvated forms. The term ‘solvate’ is used herein to describe a molecular complex comprising the compound of the invention, or a pharmaceutically acceptable salt thereof, and one or more pharmaceutically acceptable solvent molecules, for example, ethanol. The term ‘hydrate’ is employed when said solvent is water.

In addition, the compounds of Formula I may also include other solvates of such compounds which are not necessarily pharmaceutically acceptable solvates, which may be useful as intermediates for one or more of the following: 1) preparing compounds of Formula I; 2) purifying compounds of Formula I; 3) separating enantiomers of compounds of Formula I; or 4) separating diastereomers of compounds of Formula I.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together—see Chem Commun, 17; 1889-1896, by O. Almarsson and M. J. Zaworotko (2004). For a general review of multi-component complexes, see J Pharm Sci, 64(8), 1269-1288, by Haleblian (August 1975).

### Solid Form

The compounds of the invention may exist in a continuum of solid states ranging from amorphous to crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution) and consists of two dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

### Stereoisomers

Compounds of the invention may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, compounds of the invention containing one or more asymmetric carbon atoms may exist as two or more stereoisomers.

The pharmaceutically acceptable salts of compounds of the invention may also contain a counterion which is optically active (e.g., d-lactate or l-lysine) or racemic (e.g., dl-tartrate or dl-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Diastereomeric mixture may be separated by chromatography, fractional crystallization, or by using both of said techniques, and one or both of the diastereoisomers converted to the corresponding pure enantiomer(s) by means well known to a skilled person. Chiral compounds of the invention (and chiral precursors thereof) may be obtained in enantiomerically-enriched form using chromatography, typically HPLC Concentration of the eluate affords the enriched mixture. Chiral chromatography using sub- and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

### Tautomerism

Where structural isomers are interconvertible via a low energy barrier, tautomeric isomerism (‘tautomerism’) may occur. This may take the form of proton tautomerism in compounds of the invention containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the invention have been drawn herein in a single tautomeric form, all possible tautomeric forms are included within the scope of the invention.

### Isotopes

The present invention includes all pharmaceutically acceptable isotopically-labeled compounds of the invention wherein one or more atoms are replaced by atoms having the same atomic number, but an atomic mass or mass number different from the atomic mass or mass number which predominates in nature. The specific sites for deuteration of the compounds of Formula I are those where the variables Y^{1a-2a} and Y^{2a-2i} are present.

Examples of isotopes suitable for inclusion in the compounds of the invention may include isotopes of hydrogen, such as ²H (D, deuterium as described above) and ³H (T, tritium), carbon, such as ¹¹C, ¹³C and ¹⁴C, chlorine, such as ³⁶Cl, fluorine, such as ¹⁸F, nitrogen, such as ¹³N and ¹⁵N, and oxygen, such as ¹⁵O, ¹⁷O and ¹⁸O.

Certain isotopically-labelled compounds of the invention, for example those incorporating a radioactive isotope, are useful in one or both of drug or substrate tissue distribution studies. The radioactive isotopes, such as, tritium and ¹⁴C are particularly useful for this purpose in view of their ease of incorporation and ready means of detection. Substitution with positron emitting isotopes, such as, ¹¹C ¹⁸F, ¹⁵O and ¹³N, may be useful in Positron Emission Topography (PET) studies for examining substrate receptor occupancy. Substitution with deuterium may afford certain therapeutic advantages resulting from greater metabolic stability, for example increased in vivo half-life, reduced dosage requirements, reduced CYP450 inhibition (competitive or time dependent), or an improvement in therapeutic index or tolerability.

In the embodiments of the invention, the disclosure provides deuterium-labeled (or deuterated) compounds, where the formula and variables of such compounds are each and independently as described herein. “Deuterated” means that at least one of the atoms specified by variables Y^{1a-2a} and Y^{2a-2i} in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds of the invention may be defined by the deuterium enrichment factor. It is understood that one or more deuterium may exchange with hydrogen under physiological conditions.

In some embodiments, the deuterium compound is selected from Example 1 and any one of the compounds set forth in Table 1 shown in the Examples section.

In the embodiments, one or more hydrogen atoms on certain metabolic sites (i.e. on the Y^{1a-2a} and Y^{2a-2i} sites) compounds of the invention are deuterated.

Isotopically-labeled compounds of the invention may generally be prepared by conventional techniques known to those skilled in the art or by processes analogous to those described in the accompanying Examples and Preparations using an appropriate isotopically-labeled reagent in place of a corresponding non-labeled reagent.

Pharmaceutically acceptable solvates in accordance with the invention include those wherein the solvent of crystallization may be isotopically substituted, e.g., D₂O, d₆-acetone, d₆-DMSO.

### Metabolites

Also included within the scope of the invention are active metabolites of compounds of the invention, that is, compounds formed in vivo upon administration of the drug, often by oxidation or dealkylation. Some examples of metabolites in accordance with the invention include, but are not limited to,
(i) where the compound of the invention contains an alkyl group, a hydroxyalkyl derivative thereof (—CH>—COH):
(ii) where the compound of the invention contains an alkoxy group, a hydroxy derivative thereof (—OR->—OH);
(iii) where the compound of the invention contains an amide group, a carboxylic acid derivative thereof (—CONH₂—>COOH); and

Other routes of conjugative metabolism exist. These pathways are frequently known as Phase 2 metabolism and include, for example, sulfation or acetylation. Other functional groups, such as NH groups, may also be subject to conjugation.

### Pharmaceutical Compositions

In another embodiment, the invention comprises pharmaceutical compositions. For pharmaceutical composition purposes, the compound per se (a compound of Formula I) will simply be referred to as the compounds of the invention.

The compositions of this invention may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the invention. In another embodiment, the oral administration may be in a powder or granule form. In another embodiment, the oral dosage form is sub-lingual, such as, for example, a lozenge. In such solid dosage forms, the compounds of the invention are ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the invention comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

In another embodiment, the invention comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the compound of this invention is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the invention comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; Rowe, Raymond C. Handbook of Pharmaceutical Excipients. Chicago, Pharmaceutical Press, 2005; Stahl, P. Heinrich and Camilli G. Wermuth, Eds. Handbook of Pharmaceutical Salts: Properties, Selection, and Use. New York: Wiley-VCH, 2011; and Brittain, Harry G., Ed. Polymorphism in Pharmaceutical Solids. New York: Informa Healthcare USA, Inc., 2016.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

For oral administration, the compositions may be provided in the form of tablets or capsules containing 0.01, 0.05, 0.1, 0.5, 1.0, 2.5, 5.0, 10.0, 15.0, 25.0, 50.0, 75.0, 100, 125, 150, 175, 200, 250, 300, 350, 400, 450, 500, 550, 600, 650, 700, 750, 800, 850, 900, 950 or 1000 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Dosing regimens may depend on the route of administration, dose scheduling, and use of flat-dose, body surface area or weight-based dosing. For example, for weight-based dosing, intravenously doses may range from about 0.01 to about 10 mg/kg/minute during a constant rate infusion.

Liposome containing compounds of the invention may be prepared by methods known in the art (See, for example, Chang, H. I.; Yeh, M. K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Compounds of the invention may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the invention, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the invention are generally placed into a container having a sterile access port, for example, an intravenous solution bag or vial having a stopper pierceable by a hypodermic injection needle.

Suitable emulsions may be prepared using commercially available fat emulsions, such as a lipid emulsions comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a pH in the range of 5.5 to 8.0.

For example, the emulsion compositions may be those prepared by mixing a compound of the invention with a lipid emulsions comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug product intermediate (DPI) is a partly processed material that must undergo further processing steps before it becomes bulk drug product. Compounds of the invention may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. In one embodiment, the drug product intermediate contains a compound of the invention isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. In one embodiment amorphous solid dispersions comprise a compound of the invention and a polymer excipient. Other excipients as well as concentrations of said excipients and the compound of the invention are well known in the art and are described in standard textbooks. See, for example, “Amorphous Solid Dispersions Theory and Practice” by Navnit Shah et al.

### Administration and Dosing

Typically, a compound of the invention is administered in an amount effective to treat a condition as described herein such as a coronavirus infection such as COVID-19. The compounds of the invention may be administered as compound per se. For administration and dosing purposes, the compound per se will simply be referred to as the compounds of the invention.

The compounds of the invention are administered by any suitable route in the form of a pharmaceutical composition adapted to such a route, and in a dose effective for the treatment intended. The compounds of the invention may be administered orally, rectally, vaginally, parenterally, topically, intranasally, or by inhalation.

The compounds of the invention may be administered orally. Oral administration may involve swallowing, so that the compound enters the gastrointestinal tract, or buccal or sublingual administration may be employed by which the compound enters the bloodstream directly from the mouth.

In another embodiment, the compounds of the invention may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the compounds of the invention may also be administered topically to the skin or mucosa, that is, dermally or transdermally. In another embodiment, the compounds of the invention may also be administered intranasally or by inhalation. In another embodiment, the compounds of the invention may be administered rectally or vaginally. In another embodiment, the compounds of the invention may also be administered directly to the eye or ear.

The dosage regimen for the compounds of the invention or compositions containing said compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the invention is typically from about 0.01 to about 100 mg/kg (i.e., mg compound of the invention per kg body weight) for the treatment of the indicated conditions discussed herein. In another embodiment, total daily dose of the compound of the invention is from about 0.1 to about 50 mg/kg, and in another embodiment, from about 0.5 to about 30 mg/kg. It is not uncommon that the administration of the compounds of the invention will be repeated a plurality of times in a day (typically no greater than 4 times). Multiple doses per day typically may be used to increase the total daily dose, if desired. Representative dosing of the compounds of Formula I include oral dosing once or twice a day using an oral dosage form such as a tablet.

### Therapeutic Methods and Uses

The compounds of the invention may inhibit viral proteases, particularly coronavirus viral proteases such as the 3CL (Mpro) protease of SARS-CoV-2 which is the causative virus of COVID-19. The compounds of the invention may reduce the viral load in a patient, particularly coronavirus viral loads such as SARS-CoV-2 (COVID-19), SARS-CoV, MERS-Cov, HCoV-229E, HCoV-NL63, HCoV-OC43, HCoV-HKU1 viral loads. The compounds of the invention may inhibit the activity of the main viral protease and may be useful in the treatment, prevention, suppression and amelioration of viral infections including coronavirus infections such as SARS-CoV-2 (COVID-19), SARS-CoV, MERS-Cov, HCoV-229E, HCoV-NL63, HCoV-OC43, HCoV-HKU1 infections and the COVID-19 infection related disorder long COVID.

### Co-Administration

The compounds of the invention can be used alone, or in combination with one or more other therapeutic agents. The invention provides any of the uses, methods or compositions as defined herein wherein the compound of the invention is used in combination with one or more other therapeutic agent discussed herein.

The compounds of the present invention can be used in the methods of the invention in combination with other drugs. For example, dosing a SARS-CoV-2 coronavirus-infected patient (i.e., a patient with COVID-19) with the SARS-CoV-2 coronavirus 3CL protease inhibitor of the invention and nirmatrelvir, molnupirivir, remdesivir, tocilizumab, anakinra, baricitinib, vilobelimab, sabizabulin, an interferon, such as interferon lambda, interferon alpha, or a pegylated interferon, such as PEG-Intron or Pegasus, may provide a greater clinical benefit than dosing either the interferon, pegylated interferon or the SARS-CoV-2 coronavirus inhibitor alone. Other additional agents that can be used in the methods of the present invention include agents approved for treatment of viral infections such as agents approved for treatment of a SARS-CoV-2 infection. Examples of greater clinical benefits could include a larger reduction in COVID-19 symptoms, a faster time to alleviation of symptoms, reduced lung pathology, a larger reduction in the amount of SARS-CoV-2 coronavirus in the patient (viral load), and decreased mortality.

The SARS-CoV-2 coronavirus infects cells which express P-glycoprotein. Some of the SARS-CoV-2 coronavirus 3CL protease inhibitors of the invention are P-glycoprotein substrates. Compounds which inhibit the SARS-CoV-2 coronavirus which are also P-glycoprotein substrates may be dosed with a P-glycoprotein inhibitor. Examples of P-glycoprotein inhibitors are verapamil, vinblastine, ketoconazole, nelfinavir, ritonavir or cyclosporine. The P-glycoprotein inhibitors act by inhibiting the efflux of the SARS-CoV-2 coronavirus inhibitors of the invention out of the cell. The inhibition of the P-glycoprotein-based efflux will prevent reduction of intracellular concentrations of the SARS-CoV-2 coronavirus inhibitor due to P-glycoprotein efflux. Inhibition of the P-glycoprotein efflux will result in larger intracellular concentrations of the SARS-CoV-2 coronavirus inhibitors. Dosing a SARS-CoV-2 coronavirus-infected patient with the SARS-CoV-2 coronavirus 3CL protease inhibitors of the invention and a P-glycoprotein inhibitor may lower the amount of SARS-CoV-2 coronavirus 3CL protease inhibitor required to achieve an efficacious dose by increasing the intracellular concentration of the SARS-CoV-2 coronavirus 3CL protease inhibitor.

Among the agents that may be used to increase the exposure of a mammal to a compound of the present invention are those that can act as inhibitors of at least one isoform of the cytochrome P450 (CYP450) enzymes. The isoforms of CYP450 that may be beneficially inhibited include, but are not limited to CYP1A2, CYP2D6, CYP2C9, CYP2C19 and CYP3A4. The compounds used in the methods of the invention include compounds that may be CYP3A4 substrates and are metabolized by CYP3A4. Dosing a SARS-CoV-2 coronavirus-infected patient with a SARS-CoV-2 coronavirus inhibitor which is a CYP3A4 substrate, such as SARS-CoV-2 coronavirus 3CL protease inhibitor, and a CYP3A4 inhibitor, such as ritonavir, nelfinavir or delavirdine, will reduce the metabolism of the SARS-CoV-2 coronavirus inhibitor by CYP3A4. This will result in reduced clearance of the SARS-CoV-2 coronavirus inhibitor and increased SARS-CoV-2 coronavirus inhibitor plasma concentrations. The reduced clearance and higher plasma concentrations may result in a lower efficacious dose of the SARS-CoV-2 coronavirus inhibitor.

Additional therapeutic agents that can be used in combination with the SARS-CoV-2 inhibitors in the methods of the present invention include the following:

PLpro inhibitors, Apilomod, EIDD-2801, Ribavirin, Valganciclovir, β-Thymidine, Aspartame, Oxprenolol, Doxycycline, Acetophenazine, Iopromide, Riboflavin, Reproterol, 2,2′-Cyclocytidine, Chloramphenicol, Chlorphenesin carbamate, Levodropropizine, Cefamandole, Floxuridine, Tigecycline, Pemetrexed, L(+)-Ascorbic acid, Glutathione, Hesperetin, Ademetionine, Masoprocol, Isotretinoin, Dantrolene, Sulfasalazine Anti-bacterial, Silybin, Nicardipine, Sildenafil, Platycodin, Chrysin, Neohesperidin, Baicalin, Sugetriol-3,9-diacetate, (−)-Epigallocatechin gallate, Phaitanthrin D, 2-(3,4-Dihydroxyphenyl)-2-[[2-(3,4-dihydroxyphenyl)-3,4-dihydro-5,7-dihydroxy-2H-1-benzopyran-3-yl]oxy]-3,4-dihydro-2H-1-benzopyran-3,4,5,7-tetrol, 2,2-di(3-indolyl)-3-indolone, (S)-(1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl-2-amino-3-phenylpropanoate, Piceatannol, Rosmarinic acid, and Magnolol.

3CLpro inhibitors, Lymecycline, Chlorhexidine, Alfuzosin, Cilastatin, Famotidine, Almitrine, Progabide, Nepafenac, Carvedilol, Amprenavir, Tigecycline, Montelukast, Carminic acid, Mimosine, Flavin, Lutein, Cefpiramide, Phenethicillin, Candoxatril, Nicardipine, Estradiol valerate, Pioglitazone, Conivaptan, Telmisartan, Doxycycline, Oxytetracycline, (1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl5-((R)-1,2-dithiolan-3-yl) pentanoate, Betulonal, Chrysin-7-O-β-glucuronide, Andrographiside, (1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl 2-nitrobenzoate, 2β-Hydroxy-3,4-seco-friedelolactone-27-oic acid (S)-(1S,2R,4aS,5R,8aS)-1-Formamido-1,4a-dimethyl-6-methylene-5-((E)-2-(2-oxo-2,5-dihydrofuran-3-yl)ethenyl) decahydronaphthalen-2-yl-2-amino-3-phenylpropanoate, Isodecortinol, Cerevisterol, Hesperidin, Neohesperidin, Andrograpanin, 2-((1R,5R,6R,8aS)-6-Hydroxy-5-(hydroxymethyl)-5,8a-dimethyl-2-methylenedecahydronaphthalen-1-yl)ethyl benzoate, Cosmosiin, Cleistocaltone A,2,2-Di(3-indolyl)-3-indolone, Biorobin, Gnidicin, Phyllaemblinol, Theaflavin 3,3′-di-O-gallate, Rosmarinic acid, Kouitchenside I, Oleanolic acid, Stigmast-5-en-3-ol, Deacetylcentapicrin, and Berchemol.

RdRp inhibitors, Valganciclovir, Chlorhexidine, Ceftibuten, Fenoterol, Fludarabine, Itraconazole, Cefuroxime, Atovaquone, Chenodeoxycholic acid, Cromolyn, Pancuronium bromide, Cortisone, Tibolone, Novobiocin, Silybin, Idarubicin Bromocriptine, Diphenoxylate, Benzylpenicilloyl G, Dabigatran etexilate, Betulonal, Gnidicin, 2β,30β-Dihydroxy-3,4-seco-friedelolactone-27-lactone, 14-Deoxy-11,12-didehydroandrographolide, Gniditrin, Theaflavin 3,3′-di-O-gallate, (R)-((1R,5aS,6R,9aS)-1,5a-Dimethyl-7-methylene-3-oxo-6-((E)-2-(2-oxo-2,5-dihydrofuran-3-yl)ethenyl)decahydro-1H-benzo[c]azepin-1-yl)methyl2-amino-3-phenylpropanoate, 2β-Hydroxy-3,4-seco-friedelolactone-27-oic acid, 2-(3,4-Dihydroxyphenyl)-2-[[2-(3,4-dihydroxyphenyl)-3,4-dihydro-5,7-dihydroxy-2H-1-benzopyran-3-yl]oxy]-3,4-dihydro-2H-1-benzopyran-3,4,5,7-tetrol, Phyllaemblicin B, 14-hydroxycyperotundone, Andrographiside, 2-((1R,5R,6R,8aS)-6-Hydroxy-5-(hydroxymethyl)-5,8a-dimethyl-2-methylenedecahydro naphthalen-1-yl)ethyl benzoate, Andrographolide, Sugetriol-3,9-diacetate, Baicalin, (1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl 5-((R)-1,2-dithiolan-3-yl)pentanoate, 1,7-Dihydroxy-3-methoxyxanthone, 1,2,6-Trimethoxy-8-[(6-O-β-D-xylopyranosyl-β-D-glucopyranosyl)oxy]-9H-xanthen-9-one, and 1,8-Dihydroxy-6-methoxy-2-[(6-O-β-D-xylopyranosyl-β-D-glucopyranosyl)oxy]-9H-xanthen-9-one, 8-(p6-D-Glucopyranosyloxy)-1,3,5-trihydroxy-9H-xanthen-9-one,

Additional therapeutic agents that can be used in the methods of the invention include Diosmin, Hesperidin, MK-3207, Venetoclax, Dihydroergocristine, Bolazine, R428, Ditercalinium, Etoposide, Teniposide, UK-432097, Irinotecan, Lumacaftor, Velpatasvir, Eluxadoline, Ledipasvir, Lopinavir/Ritonavir+Ribavirin, Alferon, and prednisone. Other additional agents useful in the methods of the present invention include dexamethasone, azithromycin and remdesivir as well as boceprevir, umifenovir and favipiravir.

Other additional agents that can be used in the methods of the present invention include α-ketoamides compounds designated as 11r, 13a and 13b, shown below, as described in Zhang, L.; Lin, D.; Sun, X.; Rox, K.; Hilgenfeld, R.; X-ray Structure of Main Protease of the Novel Coronavirus SARS-CoV-2 Enables Design of α-Ketoamide Inhibitors; bioRxiv preprint doi: https://doi.org/10.1101/2020.02.17.952879

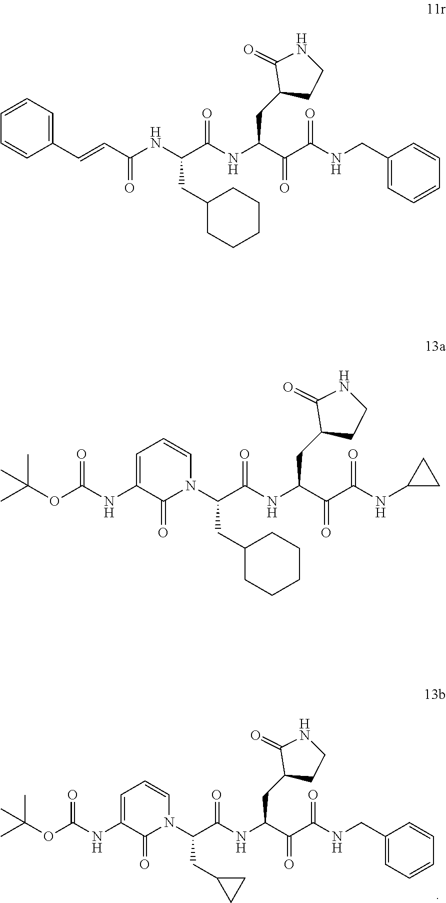 [SMILES: O=C(/C=C/c1ccccc1)N[C@@H](CC1CCCCC1)C(=O)N[C@@H](C[C@@H]1CCNC1=O)C(=O)C(=O)NCc1ccccc1 ; CC(C)(C)OC(=O)Nc1cccn([C@@H](CC2CCCCC2)C(=O)N[C@@H](C[C@@H]2CCNC2=O)C(=O)C(=O)NC2CC2)c1=O ; CC(C)(C)OC(=O)Nc1cccn([C@@H](CC2CC2)C(=O)N[C@@H](C[C@@H]2CCNC2=O)C(=O)C(=O)NCc2ccccc2)c1=O]

Additional agents that can be used in the methods of the present invention include RIG 1 pathway activators such as those described in U.S. Pat. No. 9,884,876.

Other additional therapeutic agents include protease inhibitors such as those described in Dai W. Zhang B, Jiang X-M, et al Structure-based design of antiviral drug candidates targeting the SARS-CoV-2 main protease Science. 2020; 368(6497):1331-1335 including compounds such as the compound shown below and a compound designated as DC402234

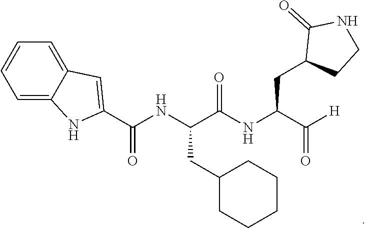 [SMILES: O=C[C@H](C[C@@H]1CCNC1=O)NC(=O)[C@H](CC1CCCCC1)NC(=O)c1cc2ccccc2[nH]1]

Another embodiment of the present invention is a method of treating COVID-19 in a patient wherein in addition to administering a compound of the present invention (i.e. a compound of Formula I or Ia-Id or a solvate or hydrate thereof an additional agent is administered and the additional agent is selected from antivirals such as remdesivir, galidesivir, favilavir/avifavir, molnupiravir (MK-4482/EIDD 2801), AT-527, AT-301, BLD-2660, favipiravir, camostat, SLV213 emtrictabine/tenofivir, clevudine, dalcetrapib, boceprevir, PBI-0451, EDP-235 and ABX464, glucocorticoids such as dexamethasone and hydrocortisone, convalescent plasma, a recombinant human plasma such as gelsolin (Rhu-p65N), monoclonal antibodies such as regdanvimab (Regkirova), ravulizumab (Ultomiris), VIR-7831/VIR-7832, BRII-196/BRII-198, COVI-AMG/COVI DROPS (STI-2020), bamlanivimab (LY-CoV555), mavrilimab, leronlimab (PRO140), AZD7442, lenzilumab, infliximab, adalimumab, JS 016, STI-1499 (COVIGUARD), lanadelumab (Takhzyro), canakinumab (Ilaris), gimsilumab and otilimab, antibody cocktails such as casirivimab/imdevimab (REGN-Cov2), recombinant fusion protein such as MK-7110 (CD24Fc/SACCOVID), anticoagulants such as heparin and apixaban, IL-6 receptor agonists such as tocilizumab (Actemra) and sarilumab (Kevzara), PIKfyve inhibitors such as apilimod dimesylate, RIPK1 inhibitors such as DNL758, DC402234, VIP receptor agonists such as PB1046, SGLT2 inhibitors such as dapaglifozin, TYK inhibitors such as abivertinib, kinase inhibitors such as ATR-002, bemcentinib, acalabrutinib, losmapimod, baricitinib and tofacitinib, H2 blockers such as famotidine, anthelmintics such as niclosamide, furin inhibitors such as diminazene.

The term “SARS-CoV-2 inhibiting agent” means any SARS-CoV-2-related coronavirus 3C-like protease inhibitor compound described herein or a pharmaceutically acceptable salt, hydrate, prodrug, active metabolite or solvate thereof or a compound which inhibits replication of SARS-CoV-2 in any manner.

The term “interfering with or preventing” SARS-CoV-2-related coronavirus (“SARS-CoV-2”) viral replication in a cell means to reduce SARS-CoV-2 replication or production of SARS-CoV-2 components necessary for progeny virus in a cell treated with a compound of this invention as compared to a cell not being treated with a compound of this invention. Simple and convenient assays to determine if SARS-CoV-2 viral replication has been reduced include an ELISA assay for the presence, absence, or reduced presence of anti-SARS-CoV-2 antibodies in the blood of the subject (Nasoff, et al., PNAS 88:5462-5466, 1991), RT-PCR (Yu, et al., in Viral Hepatitis and Liver Disease 574-577, Nishioka, Suzuki and Mishiro (Eds.); Springer-Verlag, Tokyo, 1994). Such methods are well known to those of ordinary skill in the art. Alternatively, total RNA from transduced and infected “control” cells can be isolated and subjected to analysis by dot blot or northern blot and probed with SARS-CoV-2-specific DNA to determine if SARS-CoV-2 replication is reduced. Alternatively, reduction of SARS-CoV-2 protein expression can also be used as an indicator of inhibition of SARS-CoV-2 replication. A greater than fifty percent reduction in SARS-CoV-2 replication as compared to control cells typically quantitates a prevention of SARS-CoV-2 replication.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration.

The phrases “concurrent administration,” “co-administration,” “simultaneous administration,” “sequential administration” and “administered simultaneously” mean that the compounds are administered in combination.

A compound of the invention and the one or more other therapeutic agents may be administered as a fixed or non-fixed combination of the active ingredients. The term “fixed combination” means a compound of the invention, or a pharmaceutically acceptable salt thereof, and the one or more therapeutic agents, are both administered to a subject simultaneously in a single composition or dosage. The term “non-fixed combination” means that a compound of the invention, or a pharmaceutically acceptable salt thereof, and the one or more therapeutic agents are formulated as separate compositions or dosages such that they may be administered to a subject in need thereof simultaneously or sequentially with variable intervening time limits, wherein such administration provides effective levels of the two or more compounds in the body of the subject.

In one embodiment, the compounds of this invention are administered in combination with additional therapeutic agents useful in treatment of viral infections including the pharmaceutically acceptable salts of the specifically named agents and the pharmaceutically acceptable solvates of said agents and salts.

These agents and compounds of the invention can be combined with pharmaceutically acceptable vehicles such as saline, Ringer's solution, dextrose solution, and the like. The particular dosage regimen, i.e., dose, timing and repetition, will depend on the particular individual and that individual's medical history.

### Kits

Another aspect of the invention provides kits comprising the compound of the invention or pharmaceutical compositions comprising the compound of the invention. A kit may include, in addition to the compound of the invention or pharmaceutical composition thereof, diagnostic or therapeutic agents. A kit may also include instructions for use in a diagnostic or therapeutic method. In some embodiments, the kit includes the compound or a pharmaceutical composition thereof and a diagnostic agent. In other embodiments, the kit includes the compound or a pharmaceutical composition thereof and one or more therapeutic agents, such as nirmatrelvir or molnupirivir.

In yet another embodiment, the invention comprises kits that are suitable for use in performing the methods of treatment described herein. In one embodiment, the kit contains a first dosage form comprising one or more of the compounds of the invention in quantities sufficient to carry out the methods of the invention. In another embodiment, the kit comprises one or more compounds of the invention in quantities sufficient to carry out the methods of the invention and a container for the dosage and a container for the dosage.

### Synthetic Methods

Compounds of the present invention may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein, are related to, or may be derived from compounds in which one or more of the scientific interest or commercial need has occurred. Accordingly, such compounds may be one or more of 1) commercially available; 2) reported in the literature or 3) prepared from other commonly available substances by one skilled in the art using materials which have been reported in the literature.

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compounds of the invention. It will be further appreciated that it may be necessary or desirable to carry out the transformations in a different order from that described in the schemes, or to modify one or more of the transformations, to provide the desired compound of the invention.

In the preparation of compounds of the invention it is noted that some of the preparation methods useful for the preparation of the compounds described herein may require protection of remote functionality (e.g., a primary amine, secondary amine, carboxyl, etc. in a precursor of a compound of the invention). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see March's Advanced Organic Chemistry: Reactions, Mechanisms, and Structure 8th Edition.

For example, if a compound contains a amine or carboxylic acid functionality, such functionality may interfere with reactions at other sites of the molecule if left unprotected. Accordingly, such functionalities may be protected by an appropriate protecting group (PG) which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and may typically be removed without chemically altering other functionality in a compound of the invention.

### General Experimental Details

In the non-limiting Examples and Preparations that illustrate the invention and that are set out in the description, and in the following Schemes, the following the abbreviations, definitions and analytical procedures may be referred to:

Mass spectrometry data are reported from LCMS analyses. Mass spectrometry (MS) was performed via atmospheric pressure chemical ionization (APCI), electrospray ionization (ESI), electron impact ionization (EI) or electron scatter ionization (ES) sources. Proton nuclear magnetic spectroscopy (¹H NMR) chemical shifts are given in parts per million downfield from tetramethylsilane and were recorded on 300, 400, 500, or 600 MHz Varian, Bruker, or Jeol spectrometers. Chemical shifts are expressed in parts per million (ppm, 6) referenced to the deuterated solvent residual peaks (chloroform, 7.26 ppm; CD₂HOD, 3.31 ppm; acetonitrile-d₂, 1.94 ppm; dimethyl sulfoxide-d₅, 2.50 ppm; DHO, 4.79 ppm). The multiplicity of a signal is designated by the following abbreviations: [insert abbreviations, such as “s, singlet; d, doublet; t, triplet; q, quartet; dd, doublet of doublets; dt, doublet of triplets; tt, triplet of triplets; br, broad; m, multiplet” here. All observed coupling constants, J, are reported in Hertz (Hz). Exchangeable protons are not always observed.

LCMS data were acquired on an Agilent 1100 Series instrument with a Leap Technologies autosampler, Gemini C18 columns, acetonitrile/water gradients, and either trifluoroacetic acid, formic acid, or ammonium hydroxide modifiers. The column eluate was analyzed using a Waters ZQ mass spectrometer scanning in both positive and negative ion modes from 100 to 1200 Da. Other similar instruments were also used. HPLC data were generally acquired on an Agilent 1100 Series instrument, using the columns indicated, acetonitrile/water gradients, and either trifluoroacetic acid or ammonium hydroxide modifiers.

### Abbreviations

aq is aqueous;
Boc is tert-butoxycarbonyl;
br is broad;
° C. is degrees celcius;
CDCl₃ is deutero-chloroform;
CDI is 1,1′-carbonyldiimidazole;
δ is chemical shift;
d is doublet;
dd is doublet of doublets;
ddd is doublet of doublet of doublets;
dt is doublet of triplets;
DIPEA is N-ethyldiisopropylamine, also known as N,N-diisopropylethylamine;
DMF is N,N-dimethylformamide;
DMSO is dimethyl sulfoxide;
DMSO-d₆ is deuterodimethylsulfoxide;
EDTA is tetraethylenediamine;
g is gram;
HATU is 1-[bis(dimethylamino)methylene]-1H-1,2,3-triazolo[4,5-b]pyridinium 3-oxide hexafluorophosphate;
HPLC is high pressure liquid chromatography;
h or hr(s) is hour(s);
IPA is isopropyl alcohol;
L is liter;
LCMS is liquid chromatography mass spectrometry;
m is multiplet;
M is molar;
mM is millimolar;
μM is micromolar;
MeOD_d₄ is deuterated methanol;
MeOH is methanol;
2-MeTHF is 2-methyl tetrahydrofuran;
mg is milligram;
Hz is Hertz;
MHz is mega Hertz;
min(s) is minute(s);
mL is milliliter;
mmol is millimole;
mol is mole;
MS (m/z) is mass spectrum peak;
MTBE is tert-butyl methyl ether;
NaBH₄ is sodium borohydride;
NaB²H₄ is deutero sodium borohydride;
NMR is nuclear magnetic resonance;
pH is power of hydrogen;
ppm is parts per million;
q is quartet;
rt is room temperature;
RT is retention time;
s is singlet;
SFC is supercritical fluid chromatography;
t is triplet;
TFA is trifluoroacetic acid;
THF is tetrahydrofuran; and
μL is microliter.

The Schemes described below are intended to provide a general description of the methodology employed in the preparation of the compounds of the present invention. In the following Schemes, the general methods for the preparation of the compounds are shown either in racemic or enantioenriched form. It will be apparent to one skilled in the art that all of the synthetic transformations may be conducted in a precisely similar manner whether the materials are enantioenriched or racemic. Moreover, the resolution to the desired optically active material may take place at any desired point in the sequence using well known methods such as described herein and in the chemistry literature.

### General Methods

Unless stated otherwise, the variables in Schemes I have the same meanings as defined herein.

### Scheme I:

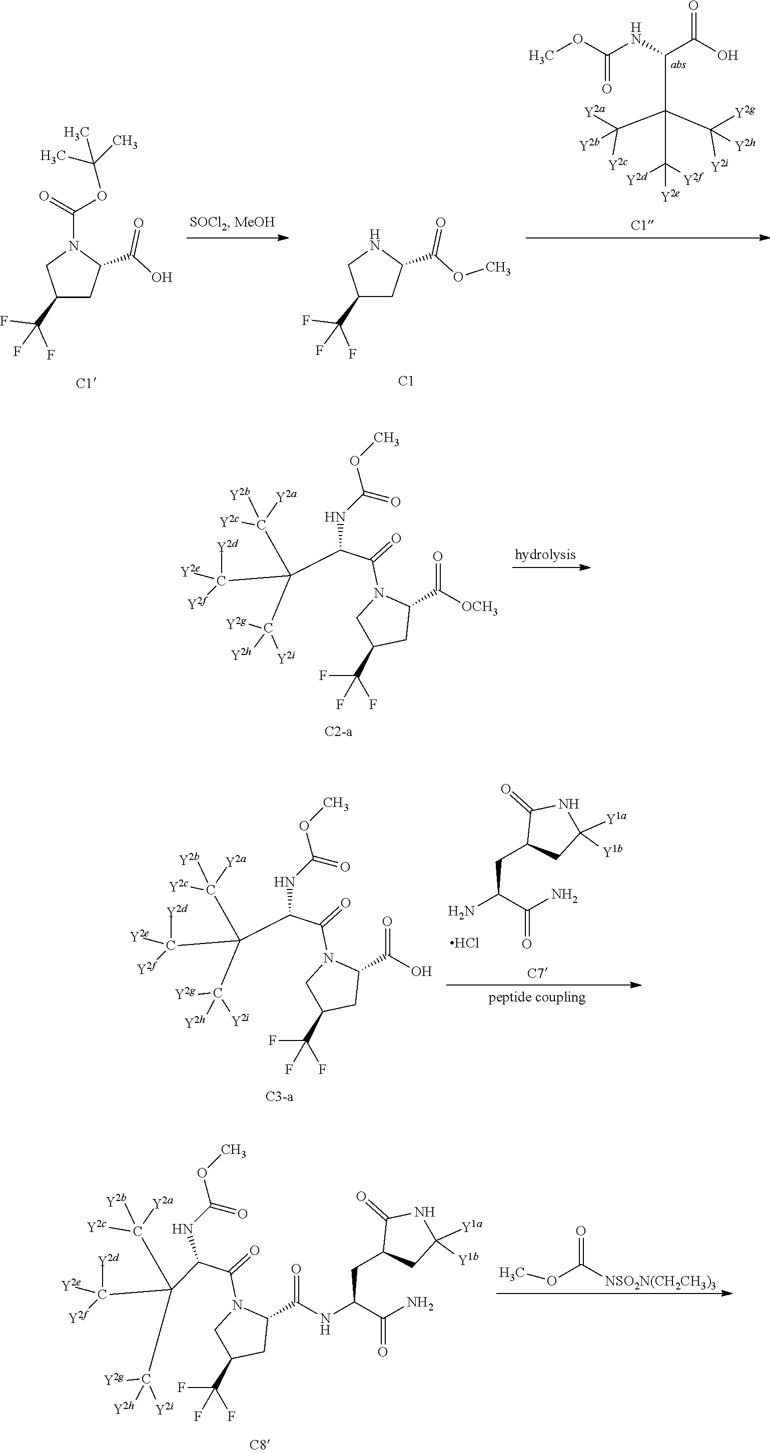

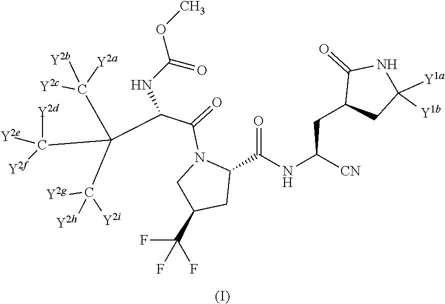

As exemplified in Scheme I, a Boc protected proline carboxylic acid compound C1′ may be treated with a thionyl chloride in methanol to provide the compound C1. Compound C1 is then coupled with the Y^{2a-i} substituted amino acid derivative C1″ in the presence of an effective peptide coupling reagent (such as HATU) in the presence of an appropriate base such as N,N-diisopropylethylamine in an appropriate solvent (such as DMF) to provide a compound C2-a. It is to be understood that other peptide coupling reagents, bases and solvents can be employed in this step peptide bond forming step. The compound C1′ contains a Boc protecting group, which may be appended or removed by additional steps in the synthetic sequence using conditions known in the art (March's Advanced Organic Chemistry: Reactions, Mechanisms, and Structure 8th Edition or Protecting Groups, 10 Georg Thieme Verlag, 1994). It is to be understood that it is possible to employ alternative amine protecting groups which provide analogs of C1′ with the amine therein protected by the other suitable amine protecting group. C2a is subjected to base catalyzed hydrolysis to provide C3a which is then coupled under peptide coupling conditions as previously described to provide C8′ which is then converted to the compound of Formula (I). Compounds at every step may be purified by standard techniques, such as column chromatography, crystallization, or reverse phase SFC or HPLC. Variables Y^{1a-b} and Y^{2a-i} are as defined in the embodiments, schemes, examples, and claims herein.

The synthetic intermediates having formulas C2-a, C3-a, C7′, C8′ as defined in the above Schemes are useful for preparing compounds of the invention and are provided as further aspects of this invention.

### EXAMPLES

In order that this invention may be better understood, the following examples are set forth. These examples are for purposes of illustration only and are not to be construed as limiting the scope of the invention in any manner.

The compounds and intermediates described below were named using the naming convention provided with ACD/Chemsketch 2020.2.1.1 (Advanced Chemistry Development, Inc. Toronto, Ontario, Canada. The naming convention provided with ACD/Chemsketch 2020.2.1.1 is well known by those skilled in the art and it is believed that the naming convention provided with ACD/Chemsketch 2020.2.1.1 generally comports with the IUPAC (International Union for Pure and Applied Chemistry) recommendations on Nomenclature of Organic Chemistry and the CAS Index rules. Unless noted otherwise, all reactants were obtained commercially without further purifications or were prepared using methods known in the literature.

### Example 1

### N-(methoxycarbonyl)-3-methyl-L-valyl-(4R)—N-{(1S)-1-cyano-2-[(3S)-2-oxo(5,5-²H₂)pyrrolidin-3-yl]ethyl}-4-(trifluoromethyl)-L-prolinamide (1)

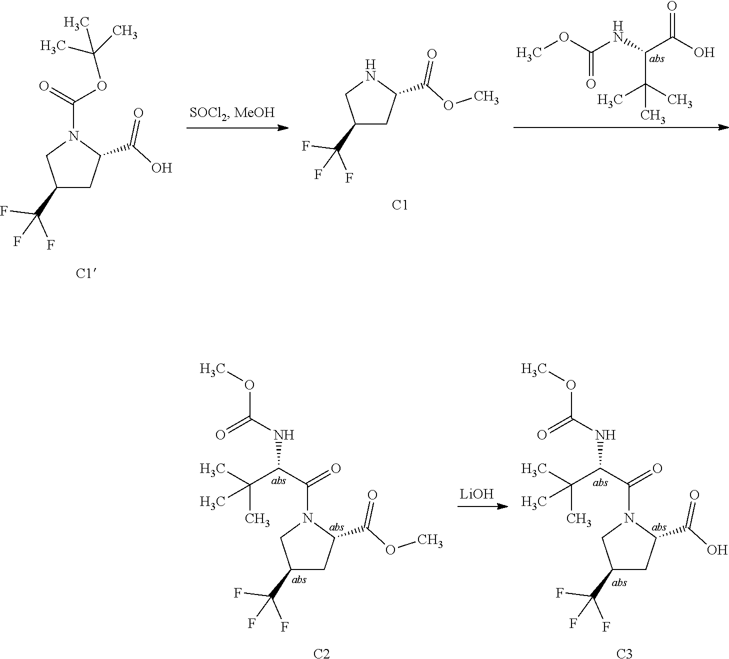

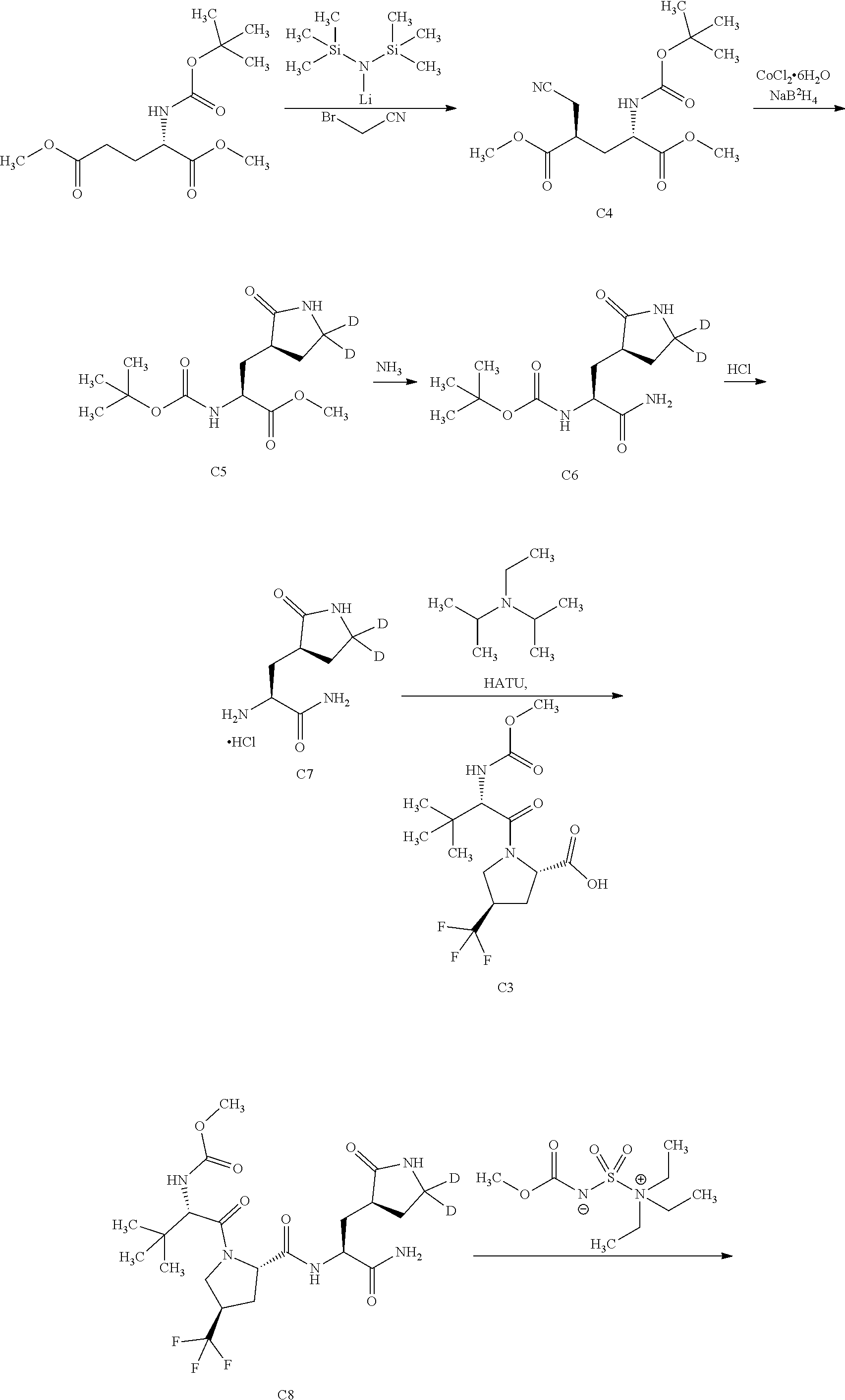

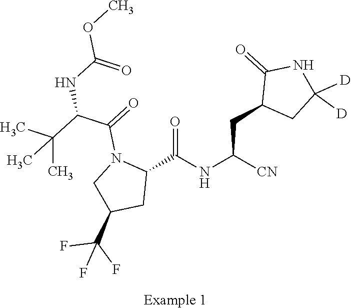

### Step 1. Synthesis of methyl (4R)-4-(trifluoromethyl)-L-prolinate (C1)

The reaction was carried out in six parallel batches: To a solution of (2S,4R)-1-(tert-butoxycarbonyl)-4-(trifluoromethyl)pyrrolidine-2-carboxylic acid (400 g, 1.41 mol) in methanol (3200 mL) was added drop wise thionyl chloride (386.42 g, 3.25 mol, 235.62 mL) over a period 1 hr, while keeping the temperature between 0° C. and 5° C. After addition, the reaction was warmed to room temperature for 16 hrs. [Note: Off-gassing was observed and was passed through 8N aqueous sodium hydroxide trap]. Each reaction was concentrated and triturated with MTBE (2 L) for 1 hr. The batches were combined and filtered. The filter cake was washed with MTBE (2 L×2), dried under vacuum to give C1 as a white solid. Yield: 1.9 kg, 8.13 mol, 96%. LCMS m/z 195.8 [M+H]+ 1H NMR (400 MHz, DMSO) δ 9.94 (s, 1H), 4.58 (dd, J=8.7, 7.1 Hz, 1H), 3.78 (s, 3H), 3.66 (dd, J=12.2, 8.9 Hz, 1H), 3.49 (qt, J=9.2, 6.9 Hz, 1H), 3.28 (dd, J=12.2, 7.2 Hz, 1H), 2.48-2.30 (m, 2H).

### Step 2. Synthesis of methyl N-(methoxycarbonyl)-3-methyl-L-valyl-(4R)-4-(trifluoromethyl)-L-prolinate (C2)

The reaction was carried out in six parallel batches: To a solution of C1 (315.5 g, 1.35 mol) in DMF (2860 mL) and acetonitrile (640 mL) was added (S)-2-((methoxycarbonyl)amino)-3,3-dimethylbutanoic acid (281.08 g, 1.49 mol), followed by N,N diisopropylethylamine (558.54 g, 4.32 mol, 752.75 mL) then portion-wise addition of HATU (564.85 g, 1.49 mol) at 0-5° C. After addition, the mixture was warmed to room temperature for 16 hrs. The reaction mixture was quenched with water (1.5 L) and extracted with ethyl acetate (3×1 L). The combined organics were washed with brine (2 L×4), dried over magnesium sulfate, filtered and concentrated under vacuum to give the crude product. Silica gel chromatography (0-25% ethyl acetate in petroleum ether) afforded C2 as white solid. Yield: 2.78 kg, 7.55 mol, 93%. MS m/z 369.4 [M+H]+ 1H NMR (400 MHz, DMSO-D6) δ 7.23 (d, J=8.6 Hz, 1H), 4.47 (dd, J=9.1, 4.9 Hz, 1H), 4.14 (d, J=8.7 Hz, 1H), 3.91 (q, J=10.6, 9.3 Hz, 2H), 3.60 (s, 3H), 3.48 (d, J=9.2 Hz, 3H), 2.33 (dt, J=15.6, 8.5 Hz, 1H), 2.16 (ddd, J=13.3, 7.9, 5.2 Hz, 1H), 0.93 (s, 9H), 0.85 (s, 1H).

### Step 3. Synthesis of N-(methoxycarbonyl)-3-methyl-L-valyl-(4R)-4-(trifluoromethyl)-L-proline (C3)

The reaction was carried out in eight parallel batches: To a solution of C2 (347.5 g, 943.40 mmol) in THF (975 mL), methanol (97.5 mL) and water (975 mL) was added lithium hydroxide monohydrate (98.97 g, 2.36 mol) in portions at 0-5° C. After addition, the reaction was warmed to room temperature for 1 h. The reaction mixture was cooled to 0° C., then the reaction was acidified by gradual addition of hydrochloric acid (3 M, 786.17 mL, 2.5 eq) to pH=−2. Extracted with ethyl acetate/2-methylTHF (V/V=2:1, 1 L×3), then combined organics were washed with saturated sodium chloride (500 mL), dried over magnesium sulfate, and filtered. The filtrates from each reaction were combined and concentrated to give crude C3 as white solid. The solid was suspended in isopropyl acetate (970 mL), n-heptane (6.05 L) was added, and the mixture was heated to 40° C. for 30 min to give a white slurry. Additional n-heptane (12.1 L) was added at 40° C., then the mixture was heated to 70° C. for 1 hr. Heat was discontinued and the mixture was allowed to cool to 20° C., then cooled to 0-5° C. for 30 min with ice water bath. The mixture was filtered. The filter cake was washed with isopropyl acetate/n-heptane (20:1, 1 L×2), dried under vacuum to give C3 as white solid. Yield: 2.5 kg, 7.06 mol, 90%. MS m/z 377.3 [M+Na]+1H NMR (400 MHz, DMSO) δ 12.74 (s, 1H), 7.25 (d, J=8.8 Hz, 1H), 4.42 (dd, J=9.0, 5.0 Hz, 1H), 4.18 (d, J=8.9 Hz, 1H), 3.95 (d, J=7.0 Hz, 2H), 3.54 (s, 3H), 2.41-2.29 (m, 1H), 2.18 (ddd, J=13.3, 7.8, 5.1 Hz, 1H), 0.98 (s, 9H), 0.90 (s, 1H).

### Step 4. Synthesis of dimethyl (4R)—N-(tert-butoxycarbonyl)-4-(cyanomethyl)-L-glutamate

Lithium hexamethyldisilazide (1M solution in tetrahydrofuran; 79.9 mL, 13.4 g, 79.9 mmol) was added dropwise to a −78° C. cooled solution of dimethyl (tert-butoxycarbonyl)-L-glutamate (10.0 g, 36.3 mmol) in tetrahydrofuran (120 mL). After stirring at −78° C. for 1.5 hours, 2-bromoacetontrile (6.54 g, 54.5 mmol) was added and the mixture was stirred for an additional 1.5 hours at −78° C. then quenched by the addition of methanol (10 mL). The reaction mixture was poured into ice water and extracted with ethyl acetate (3×60 mL). The combined extracts were washed with saturated aqueous sodium chloride solution (100 mL), dried over anhydrous sodium sulfate, filtered and concentrated in vacuo to yield a dark brown oil. Purification via silica gel chromatography (gradient 0-52% ethyl acetate in petroleum ether) afforded C4 as a yellow oil. Yield: 5.61 g, 17.85 mmol, 49%. LCMS m/z 315.1 [M+H]+. ¹H NMR (400 MHz, chloroform-d) δ 5.12 (br d, J=7.5 Hz, 1H), 4.45-4.33 (m, 1H), 3.77 (s, 3H), 3.76 (s, 3H), 2.92-2.70 (m, 3H), 2.25-2.10 (m, 2H), 1.45 (s, 9H).

### Step 5. Synthesis of methyl N-(tert-butoxycarbonyl)-3-[(3S)-2-oxo(5,5-²H₂)pyrrolidin-3-yl]-L-alaninate (C5)

A solution of C4 (3.27 g, 10.40 mmol) in tetradeuteromethanol (35 mL) was cooled to 0° C., cobalt(II) chloride (810 mg, 6.24 mmol) was added and then sodium borodeuteride (1.74 g, 41.60 mmol) was added in portions. The resulting mixture was warmed to 25° C. and stirred for 16 hours. The reaction mixture was adjusted to pH ˜4-5 by the addition of 1M aqueous hydrochloric acid solution and then concentrated in vacuo. Water (50 mL) was added to the residue and the mixture was extracted with ethyl acetate (3×15 mL). The combined extracts were washed with saturated aqueous sodium chloride solution (40 mL), dried over anhydrous sodium sulfate, filtered and concentrated in vacuo to give a green colored gum. Purification via silica gel chromatography (gradient 0-6% methanol in dichloromethane) afforded C5 as a colorless gum. Yield: 1.15 g, 3.99 mmol, 38%. LCMS m/z 289.2 [M+H]+. ¹H NMR (400 MHz, DMSO-d₆) δ 7.62 (s, 1H), 7.42 (d, J=7.9 Hz, 1H), 4.07-3.97 (m, 1H), 3.63 (s, 3H), 2.31-2.21 (m, 1H), 2.15-2.08 (m, 1H), 2.04-1.95 (m, 1H), 1.65-1.52 (m, 2H), 1.38 (s, 9H).

### Step 6. Synthesis of tert-butyl {(2S)-1-amino-1-oxo-3-[(3S)-2-oxo(5,5-²H₂)pyrrolidin-3-yl]propan-2-yl}carbamate (C6)

A solution of ammonia in methanol (7 M; 35 mL, 245 mmol) was added to a stirring solution of C5 (1.15 g, 3.99 mmol) in methanol (5 mL). After stirring for 48 hours, the reaction mixture was concentrated in vacuo. This crude material was combined with material from a similar reaction using C5 (530 mg, 1.84 mmol). The combined crude materials were reconcentrated twice from dichloromethane to afford a white solid. Purification via silica gel chromatography (gradient 0-11% methanol in dichloromethane) afforded C6 as a white solid. Combined yield: 1.30 g, 4.76 mmol, 82%. LCMS m/z 296.3 [M+Na]+. ¹H NMR (400 MHz, DMSO-d₆) δ 7.61 (br s, 1H), 7.27 (br s, 1H), 6.99 (br s, 1H), 6.90 (br d, J=8.5 Hz, 1H), 3.95-3.83 (m, 1H), 2.27-2.07 (m, 2H), 1.94-1.83 (m, 1H), 1.70-1.61 (m, 1H), 1.57-1.44 (m, 1H), 1.38 (s, 9H).

### Step 7. Synthesis of 3-[(3S)-2-oxo(5,5-²H₂)pyrrolidin-3-yl]-L-alaninamide, hydrochloride salt (C7)

To a solution of C6 (1.10 g, 4.03 mmol) in dichloromethane (6 mL) was added a solution of hydrogen chloride in 1,4-dioxane (4 M; 12 mL, 48 mmol). The reaction mixture was stirred at 0° C. for 1.5 hours, whereupon it was concentrated in vacuo to provide C7 as a white solid which was used in further chemistry without purification. Yield: 860 mg, 4.03 mmol, assumed quantitative. LCMS m/z 174.2 [M+H]+. ¹H NMR (400 MHz, DMSO-d₆) δ 8.41 (br s, 3H), 8.09 (br s, 1H), 7.93 (br s, 1H), 7.56 (br s, 1H), 3.89-3.70 (m, 1H), 2.57-2.50 (m partially obscured by DMSO peak, 1H), 2.33-2.23 (m, 1H), 2.08-1.94 (m, 1H), 1.80-1.65 (m, 2H).

### Step 8. Synthesis of N-(methoxycarbonyl)-3-methyl-L-valyl-(4R)-4-(trifluoromethyl)-L-prolyl-3-[(3S)-2-oxo(5,5-²H₂)pyrrolidin-3-yl]-L-alaninamide (C8)

O-(7-azabenzotriazol-1-yl)-N,N,N,N′-tetramethyluronium hexafluorophosphate (HATU; 11.6 g, 30.4 mmol) was added to a 0° C. solution of C7 (95 mg, 0.47 mmol) and C3 (270 mg, 0.76 mmol) in dimethylformamide (3 mL), then diisopropylethylamine (271 mg, 2.10 mmol, 13.3 mL) was added dropwise. The mixture was stirred for 16 hours at 25° C. whereupon the reaction was diluted with water (10 mL) and extracted with 10:1 dichloromethane:methanol (3×10 mL). The combined extracts were washed with 1 M aqueous hydrochloric acid solution, sodium carbonate solution and saturated aqueous sodium chloride solution, dried over anhydrous sodium sulfate, filtered, and concentrated in vacuo to give a yellow gum (350 mg). Purification via silica gel chromatography (gradient 0-10% methanol in dichloromethane) afforded C8 as a white solid. Yield: 130 mg, 0.25 mmol, 55%. LCMS m/z 510.2 [M+H]+. ¹H NMR (400 MHz, DMSO-d₆) δ 8.28 (br d, J=8.8 Hz, 1H), 7.53 (br s, 1H), 7.33 (br s, 1H), 7.27 (br d, J=8.8 Hz, 1H), 7.05 (br s, 1H), 4.54-4.47 (m, 1H), 4.28-4.21 (m, 1H), 4.15-4.11 (m, 1H), 3.96-3.88 (m, 1H), 3.52 (s, 3H), 3.18-3.15 (m, 1H), 2.50-2.40 (m, 2H), 2.29-2.20 (m, 1H), 2.15-2.08 (m, 2H), 1.98-1.89 (m, 1H), 1.63-1.57 (m, 1H), 1.52-1.44 (m, 1H), 0.94 (s, 9H).

### Step 9. Synthesis of N-(methoxycarbonyl)-3-methyl-L-valyl-(4R)—N-{(1S)-1-cyano-2-[(3S)-2-oxo(5,5-²H₂)pyrrolidin-3-yl]ethyl}-4-(trifluoromethyl)-L-prolinamide (1)

Methyl N-(triethylammoniosulfonyl)carbamate, inner salt (Burgess reagent; 152 mg, 0.64 mmol) was added to a solution of C8 (130 mg, 0.26 mmol) in dichloromethane (3 mL) and the mixture was stirred at 25° C. for 16 hours. The reaction mixture was concentrated in vacuo. Reversed-phase chromatography (Column: Boston Prime C18; Mobile phase A: 10 mM aqueous ammonium carbonate containing 0.05% ammonium hydroxide; Mobile phase B: acetonitrile; Gradient: 21% to 51% B) provided 1 as a white solid. Yield: 60 mg, 0.122 mmol, 48%. LCMS m/z 492.2 [M+H⁺]. ¹H NMR (400 MHz, chloroform-d) δ 9.03 (br d, J=8.4 Hz, 1H), 7.64 (br s, 1H), 7.27 (br d, J=8.8 Hz, 1H), 4.97-4.91 (m, 1H), 4.37-4.34 (m, 1H), 4.18-4.11 (m, 1H), 4.00-3.95 (m, 2H), 3.52 (s, 3H), 2.52-2.40 (m, partially obscured by DMSO peak, 2H), 2.35-2.23 (m, 1H), 2.19-2.03 (m, 3H), 1.74-1.62 (m, 2H), 0.94 (s, 9H).

The compounds shown in Table 1 (1-D to 5-D) are additional deuterated analogs (DA) of that can be prepared in a manner analogous to Example 1. The DAs are predicted based on the metabolic profile of the non-deuterated analog of Example 1, N-(methoxycarbonyl)-3-methyl-L-valyl-(4R)—N-{(1S)-1-cyano-2-[(3S)-2-oxopyrrolidin-3-yl]ethyl}-4-(trifluoromethyl)-L-prolinamide.

TABLE 1

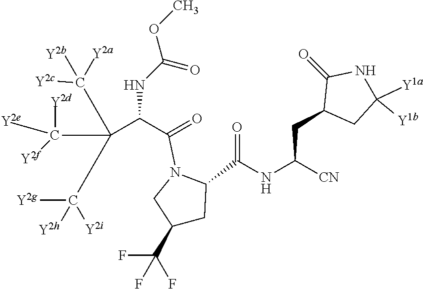

| Example | | | | | | | | | | | |
|---|---|---|---|---|---|---|---|---|---|---|---|
| Number | Y^{1a} | Y^{1b} | Y^{2a} | Y^{2b} | Y^{2c} | Y^{2d} | Y^{2e} | Y^{2f} | Y^{2g} | Y^{2h} | Y²ⁱ |
| 1-D | H | D | H | H | H | H | H | H | H | H | H |
| 2-D | H | D | D | H | H | H | H | H | H | H | H |
| 3-D | D | D | D | D | D | D | D | D | D | D | D |
| 4-D | H | H | D | D | D | D | D | D | D | D | D |
| 5-D | H | D | D | D | D | D | D | D | D | D | D |

The metabolite profile of the non-deuterated analog of Example 1, N-(methoxycarbonyl)-3-methyl-L-valyl-(4R)—N-{(1S)-1-cyano-2-[(3S)-2-oxopyrrolidin-3-yl]ethyl}-4-(trifluoromethyl)-L-prolinamide, was evaluated in liver microsomes as well as clinical studies. The metabolite profile of N-(methoxycarbonyl)-3-methyl-L-valyl-(4R)—N-{(1S)-1-cyano-2-[(3S)-2-oxopyrrolidin-3-yl]ethyl}-4-(trifluoromethyl)-L-prolinamide is comprised of oxidation and amide bond hydrolysis. Oxidation (hydroxylation) was observed at one of the positions which is designated as Y^{1a} or Y^{1b} or at one of the positions designated as Y^{2a}-Y²ⁱ in Formula I.

General methods/reviews of obtaining metabolite profile and identifying metabolites of a compound are described in: Dalvie, et al., “Assessment of Three Human in Vitro Systems in the Generation of Major Human Excretory and Circulating Metabolites,” Chemical Research in Toxicology, 2009, 22, 2, 357-368, tx8004357 (acs.org); King, R., “Biotransformations in Drug Metabolism,” Ch.3, Drug Metabolism Handbook Introduction, https://doi.org/10.1002/9781119851042.ch3; Wu, Y., et al, “Metabolite Identification in the Preclinical and Clinical Phase of Drug Development,” Current Drug Metabolish, 2021, 22, 11, 838-857, 10.2174/1389200222666211006104502; Godzien, J., et al, “Chapter Fifteen—Metabolite Annotation and Identification”.

Numerous publicly available and commercially available software tools are available to aid in the predictions of metabolic pathways and metabolites of compounds. Examples of such tools include, BioTransofrmer 3.0 (biotransformer.ca/new) which predicts the metabolic biotransformations of small molecules using a database of known metabolic reactions; MetaSite (moldiscovery.com/software/metasite/) which predicts metabolic transformations related to cytochrome P450 and flavin-containing monooxygenase mediated reactions in phase I metabolism; and Lhasa Meteor Nexus (Ihasalimited.org/products/meteor-nexus.htm) offers prediction of metabolic pathways and metabolite structures using a range of machine learning models, which covers phase I and phase II biotransformations of small molecules.

Examples 1 and 1-D through 5-D in Table 1 may afford certain therapeutic advantages resulting from greater metabolic stability, for example increased in vivo half-life, reduced dosage requirements, reduced CYP450 inhibition (competitive or time dependent), or an improvement in therapeutic index or tolerability when compared to non-deuterated analogs (compounds in which none of Y^{1a-b} and Y^{2a-i} are D).

A person with ordinary skill may make additional deuterated analogs of Example 1 and 1-D through 5-D with different combinations of the variables Y^{1a-b} and Y^{2a-i} as provided in Table 1. Such additional deuterated analogs may provide similar therapeutic advantages that may be achieved by the deuterated analogs.

Antiviral Activity from SARS-CoV-2 Infection

The ability of compounds to prevent SARS-CoV-2 coronavirus-induced cell death or cytopathic effect can be assessed via cell viability, using an assay format that utilizes luciferase to measure intracellular ATP as an endpoint. In brief, VeroE6 cells that are enriched for hACE2 expression were batched inoculated with SARS-CoV-2 (USA_WA1/2020) at a multiplicity of infection of 0.002 in a BSL-3 lab. Virus-inoculated cells were then added to assay-ready compound plates at a density of 4,000 cells/well. Following a 3-day incubation, a time at which virus-induced cytopathic effect is 95% in the untreated, infected control conditions, cell viability was evaluated using Cell Titer-Glo (Promega), according to the manufacturer's protocol, which quantitates ATP levels. Cytotoxicity of the compounds was assessed in parallel non-infected cells. Test compounds are tested either alone or in the presence of the P-glycoprotein (P-gp) inhibitor CP-100356 at a concentration of 2 μM. The inclusion of CP-100356 is to assess if the test compounds are being effluxed out of the VeroE6 cells, which have high levels of expression of P-glycoprotein. Percent effect at each concentration of test compound was calculated based on the values for the no virus control wells and virus-containing control wells on each assay plate. The concentration required for a 50% response (EC₅₀) value was determined from these data using a 4-parameter logistic model. EC₅₀ curves were fit to a Hill slope of 3 when >3 and the top dose achieved 50% effect. If cytotoxicity was detected at greater than 30% effect, the corresponding concentration data was eliminated from the EC₅₀ determination.

For cytotoxicity plates, a percent effect at each concentration of test compound was calculated based on the values for the cell-only control wells and hyamine-containing-control wells on each assay plate. The CC₅₀ value was calculated using a 4-parameter logistic model. A TI was then calculated by dividing the CC₅₀ value by the EC₅₀ value.

In the following assays data is obtained using the compound of Example 1.

### SARS-CoV-2 Coronavirus 3C Protease FRET Assay and Analysis

The proteolytic activity of the main protease, 3CLpro, of SARS-CoV-2 was monitored using a continuous fluorescence resonance energy transfer (FRET) assay. The SARS-CoV-2 3CLpro assay measures the activity of full-length SARS-CoV-2 3CL protease to cleave a synthetic fluorogenic substrate peptide with the following sequence: Dabcyl-KTSAVLQ-SGFRKME-Edans modelled on a consensus peptide (V. Grum-Tokars et al. Evaluating the 3C-like protease activity of SARS-coronavirus: recommendations for standardized assays for drug discovery. Virus Research 133 (2008) 63-73). The fluorescence of the cleaved Edans peptide (excitation 340 nm/emission 490 nm) is measured using a fluorescence intensity protocol on a Flexstation reader (Molecular Devices). The fluorescent signal is reduced in the present of PF-835231, a potent inhibitor of SARS-CoV-2 3CLpro. The assay reaction buffer contained 20 mM Tris-HCl (pH 7.3), 100 nM NaCl, 1 mM EDTA and 25 μM peptide substrate. Enzyme reactions were initiated with the addition of 15 nM SARS-CoV-2 3CL protease and allowed to proceed for 60 minutes at 23° C. Percent inhibition or activity was calculated based on control wells containing no compound (0% inhibition/100% activity) and a control compound (100% inhibition/0% activity). IC₅₀ values were generated using a four-parameter fit model using ABASE software (IDBS). Ki values were fit to the Morrison equation with the enzyme concentration parameter fixed to 15 nM, the Km parameter fixed to 14 μM and the substrate concentration parameter fixed to 25 μM using ABASE software (IDBS).

Proteolytic activity of SARS-CoV-2 Coronavirus 3CL protease is measured using a continuous fluorescence resonance energy transfer assay. The SARS-CoV-2 3CL^{pro} FRET assay measures the protease catalyzed cleavage of TAMRA-SITSAVLQSGFRKMK-(DABCYL)-OH to TAMRA-SITSAVLQ and SGFRKMK(DABCYL)-OH. The fluorescence of the cleaved TAMRA (ex. 558 nm/em. 581 nm) peptide was measured using a TECAN SAFIRE fluorescence plate reader over the course of 10 min. Typical reaction solutions contained 20 mM HEPES (pH 7.0), 1 mM EDTA, 4.0 μM FRET substrate, 4% DMSO and 0.005% Tween-20. Assays were initiated with the addition of 25 nM SARS 3CL^{pro} (nucleotide sequence 9985-10902 of the Urbani strain of SARS coronavirus complete genome sequence (NCBI accession number AY278741)). Percent inhibition was determined in duplicate at 0.001 mM level of inhibitor. Data was analyzed with the non-linear regression analysis program Kalidagraph using the equation:

$$FU = offset + ( limit ) ( 1 - {e}^{- ( kobs )} t )$$

where offset equals the fluorescence signal of the un-cleaved peptide substrate, and limit equals the fluorescence of fully cleaved peptide substrate. The kobs is the first order rate constant for this reaction, and in the absence of any inhibitor represents the utilization of substrate. In an enzyme start reaction which contains an irreversible inhibitor, and where the calculated limit is less than 20% of the theoretical maximum limit, the calculated kobs represents the rate of inactivation of coronavirus 3C protease. The slope (kobs/I) of a plot of kobs vs. [I] is a measure of the avidity of the inhibitor for an enzyme. For very fast irreversible inhibitors, kobs/I is calculated from observations at only one or two [I] rather than as a slope.
Experimental Procedure for Differentiated Normal Human Bronchial Epithelial (dNHBE) Cell Antiviral Assay

Antiviral activity of compounds was evaluated in differentiated normal human bronchial epithelial (dNHBE) cells in a BSL-3 facility. The dNHBE cells (EpiAirway) were procured from MatTek Corporation (Ashland, MA) and were grown on trans-well inserts consisting of approximately 1.2×106 cells in MatTek's proprietary culture medium (AIR-100-MM) added to the basolateral side, with the apical side exposed to a humidified 5% CO2 environment at 37° C. On day 1, dNHBE cells were infected with SARS-CoV-2 strain USA-WA1/2020 at a MOI of approximately 0.0015 50% of the cell culture infectious dose (CCID50) per cell, and compound treatment was carried out by inclusion of drug dilutions in basolateral culture media. At day 3 and day 5, virus released into the apical compartment was harvested by the addition of 0.4 ml culture media. The virus titer was then quantified by infecting 62 Vero76 cells in a standard endpoint dilution assay and virus dose that was able to infect 50% of the cell cultures (CCID50 per ml) was calculated (56). To determine the EC50 and EC90, the CCID50/ml values were normalized to that of no drug control as a percentage of inhibition and plotted against compound concentration in GraphPad Prism software by using four-parameter logistic regression.

Comparative Antiviral in Differentiated Normal Human Bronchial Epithelial (dNHBE) Cells

### Experimental for Plasma Protein Binding

Plasma protein binding was determined using a 96-well equilibrium dialysis method. Frozen human plasma (n=3 male and n=3 female, pooled) was thawed and adjusted to pH 7.4. Plasma was spiked with a final concentration of 2 μM of test compound (final organic 1% DMSO). Fraction unbound in plasma (fᵤ,p) was determined by equilibrium dialysis using an HTD 96 device assembled with 12-14 k molecular weight cutoff membranes (HTDialysis, LLC, Gales Ferry, CT). Dialysis chambers were loaded with 150 μl plasma and 150 μl phosphate buffered saline in the donor and receiver chambers, respectively. The dialysis plate was sealed with a gas-permeable membrane and stored in a 37° C. water-jacketed incubator maintained at 75% relative humidity and 5% CO2, on a 100 rpm plate shaker. After a 6-hour incubation, samples were matrix-matched and quenched by protein precipitation, followed by analysis of test compound by liquid chromatography mass spectrometry (LC-MS/MS). A set of satellite samples was included to measure stability after a 6-hour incubation. Incubations were conducted with 4-12 replicates per concentration. The unbound plasma fraction of test compounds were calculated by dividing the analyte concentration, or analyte to internal standard peak area ratio, in the buffer sample by the concentration in the donor sample, corrected for any dilution factors. All incubations had >70% analyte recovery and >70% stability in 6 h.

### Experimental for Human Liver Microsomal Stability

Test compounds (1 μM) were incubated in HLM (pool of 50 donors of mixed gender) purchased from Sekisui XenoTech (Kansas City, KS) (1 mg/ml) in potassium phosphate buffer (100 mM, pH 7.4) supplemented with MgCl₂ (3.3 mM) and β-Nicotinamide adenine dinucleotide phosphate, reduced form (NADPH) (1.3 mM) in a final volume of 400 μl. Stock solutions of test compounds were prepared in acetonitrile (final concentration of acetonitrile in the incubations were ≤0.5%). Incubations were conducted at 37° C. in triplicate. Control incubations in the absence of NADPH were also conducted in parallel in duplicate. Periodic aliquots (40 μl) of the incubation mixture at 0, 5, 10, 15, 20, 30, 45 and 60 min were removed and quenched in acetonitrile (160 ml) containing diclofenac (25 ng/ml) as internal standard. Samples from the microsomal stability assays were vortexed, centrifuged (5 min, 2300×g), and the supernatant was diluted with an equal volume of water. Samples were analyzed for depletion of test compounds by LC-MS/MS. Analyst software (Sciex, Framingham, MA) was used to measure peak areas. Peak area ratios of analyte to internal standard were calculated. Depletion half-life (t_{1/2}) and apparent intrinsic clearance (CL_{int,app}) were calculated using E-WorkBook v10 (ID Business Solutions, Guildford, Surrey, UK). The natural log of peak area ratios versus time were fitted using linear regression, and the slope (k) was converted to t_{1/2} values, where t_{1/2}=−0.693/k. To estimate CL_{int,app} in liver microsomes, the t_{1/2} for substrate depletion was scaled using the following equation (Obach et al., 1997).

$${CL}_{int . app} = \frac{0.693 \cdot ml incubation}{{t}_{1 / 2} \cdot amount of protein in incubation}$$

The following equations were used to calculate scaled intrinsic clearance (CL_{int,scaled}) and hepatic blood clearance (CL_{hep,blood}) in liver microsomes from human. The physiological constants for human were 21 g liver/kg body weight and Q of 20 ml/min/kg (human).

$${CL}_{int , scaled} = {CL}_{int , app} \cdot 45 mg microsomes / g liver \cdot g liver / kg body weight$$

$$\_CLhep . blood = \frac{Q \cdot {f}_{u . p} / {R}_{b} \cdot {CL}_{int , scaled} / {f}_{u . mic}}{Q + {f}_{u . p} / {R}_{b} \cdot {CL}_{int , scaled} / {f}_{u , mic}}$$

In biological assays used to profile compounds of the invention the Compound of Example 1 had an IC50 of 16 nM in biochemistry, Vero cell EC50=70 nM, HLM<7.2, RRCK=0.92

### nd an SF Log D=0.91.

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

We claim:

1. A compound of Formula (I) 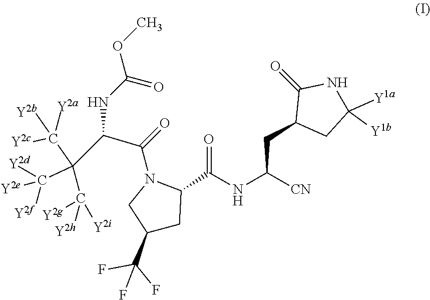
wherein
Y^{1a}, Y^{1b}, Y^{2a}, Y^{2b}, Y^{2c}, Y^{2d}, Y^{2e}, Y^{2f}, Y^{2g}, Y^{2h} and Y²ⁱ are each independently H or D; provided that at least one of Y^{1a}, Y^{1b}, Y^{2a}, Y^{2b}, Y^{2c}, Y^{2d}, Y^{2e}, Y^{2f}, Y^{2g}, Y^{2h} and Y²ⁱ is D.

2. A compound of claim 1 wherein Y^{1a} and Y^{1b} are D; and Y^{2a}, Y^{2b}, Y^{2c}, Y^{2d}, Y^{2e}, Y^{2f}, Y^{2g}, Y^{2h} and Y²ⁱ are H.

3. A compound of claim 1 wherein Y^{1a} is D; and Y^{1b}, Y^{2a}, Y^{2b}, Y^{2c}, Y^{2d}, Y^{2e}, Y^{2f}, Y^{2g}, Y^{2h} and Y²ⁱ are H.

4. A compound of claim 1 wherein Y^{2a} is D; and Y^{1a}, Y^{1b}, Y^{2b}, Y^{2c}, Y^{2d}, Y^{2e}, Y^{2f}, Y^{2g}, Y^{2h} and Y²ⁱ are H.

5. A compound of any one of claims 1 to 3 wherein Y^{2a} is D; and Y^{2b}, Y^{2c}, Y^{2d}, Y^{2e}, Y^{2f}, Y^{2g}, Y^{2h} and Y²ⁱ are H.

6. A compound of any one of claims 1 to 3 wherein Y^{2a} and Y^{2b} are D; and Y^{2c}, Y^{2d}, Y^{2e}, Y^{2f}, Y^{2g}, Y^{2h} and Y²ⁱ are H.

7. A compound of any one of claims 1 to 3 wherein Y^{2a}, Y^{2b} and Y^{2c} are D; and Y^{2d}, Y^{2e}, Y^{2f}, Y^{2g}, Y^{2h} and Y²ⁱ are H.

8. A compound of any one of claims 1 to 3 wherein Y^{2a}, Y^{2b}, Y^{2c} and Y^{2d} are D; and Y^{2e}, Y^{2f}, Y^{2g}, Y^{2h} and Y²ⁱ are H.

9. A compound of any one of claims 1 to 3 wherein Y^{2a}, Y^{2b}, Y^{2c}, Y^{2d} and Y^{2e} are D; and Y^{2f}, Y^{2g}, Y^{2h} and Y²ⁱ are H.

10. A compound of any one of claims 1 to 3 wherein Y^{2a}, Y^{2b}, Y^{2c}, Y^{2d}, Y^{2e} and Y^{2f} are D; and Y^{2g}, Y^{2h} and Y²ⁱ are H.

11. A compound of any one of claims 1 to 3 wherein Y^{2a}, Y^{2b}, Y^{2c}, Y^{2d}, Y^{2e}, Y^{2f} and Y^{2g} are D; and Y^{2h} and Y²ⁱ are H.

12. A compound of any one of claims 1 to 3 wherein Y^{2a}, Y^{2b}, Y^{2c}, Y^{2d}, Y^{2e}, Y^{2f}, Y^{2g} and Y^{2h} are D; and Y²ⁱ is H.

13. A compound of any one of claims 1 to 3 wherein Y^{2a}, Y^{2b}, Y^{2c}, Y^{2d}, Y^{2e}, Y^{2f}, Y^{2g}, Y^{2h} and Y²ⁱ are D.

14. A compound of claim 1 wherein Y^{1a} and Y^{1b} are H; and Y^{2a}, Y^{2b}, Y^{2c}, Y^{2d}, Y^{2e}, Y^{2f}, Y^{2g}, Y^{2h} and Y²ⁱ are D.

15. A compound of claim 1 wherein Y^{1a} and Y^{1b} are H; and any 2 to 7 of Y^{2a}, Y^{2b}, Y^{2c}, Y^{2d}, Y^{2e}, Y^{2f}, Y^{2g}, Y^{2h} and Y²ⁱ are D.

16. A compound of claim 1 wherein Y^{1a} is D and Y^{1b} is H; and any 2 to 7 of Y^{2a}, Y^{2b}, Y^{2c}, Y^{2d}, Y^{2e}, Y^{2f}, Y^{2g}, Y^{2h} and Y²ⁱ are D.

17. A compound of claim 1 wherein Y^{1a} and Y^{1b} are D; and any 2 to 7 of Y^{2a}, Y^{2b}, Y^{2c}, Y^{2d}, Y^{2e}, Y^{2f}, Y^{2g}, Y^{2h} and Y²ⁱ are D.

18. A compound of claim 1 having Formula (Ia) 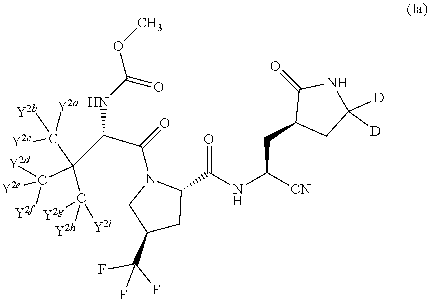
wherein 0 to 9 of Y^{2a}, Y^{2b}, Y^{2c}, Y^{2d}, Y^{2e}, Y^{2f}, Y^{2g}, Y^{2h} and Y²ⁱ are D.

19. A compound of claim 1 having Formula (Ib) 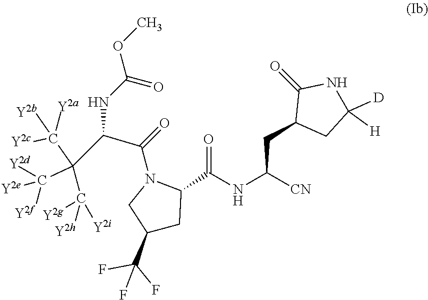
wherein 0 to 9 of Y^{2a}, Y^{2b}, Y^{2c}, Y^{2d}, Y^{2e}, Y^{2f}, Y^{2g}, Y^{2h} and Y²ⁱ are D.

20. A compound of claim 1 having Formula (Ic) 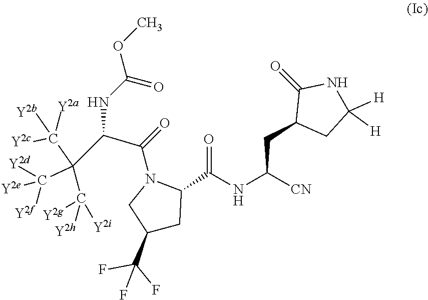
wherein 1 to 9 of Y^{2a}, Y^{2b}, Y^{2c}, Y^{2d}, Y^{2e}, Y^{2f}, Y^{2g}, Y^{2h} and Y²ⁱ are D.

21. A compound of claim 1 having Formula (Id) 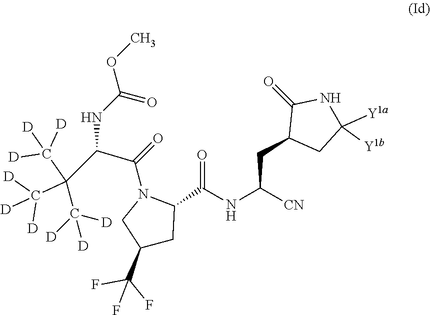
wherein Y^{1a} and Y^{1b} are each independently H or D.

22. A compound of claim 1 having the Formula 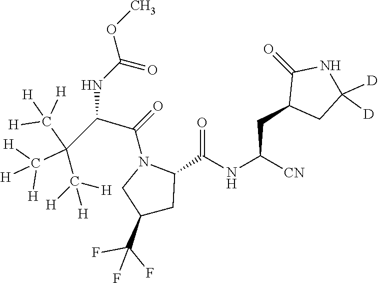 [SMILES: [2H]C1([2H])C[C@@H](C[C@@H](C#N)NC(=O)[C@@H]2C[C@@H](C(F)(F)F)CN2C(=O)[C@@H](NC(=O)OC)C(C)(C)C)C(=O)N1]
and is named N-(methoxycarbonyl)-3-methyl-L-valyl-(4R)—N-{(1S)-1-cyano-2-[(3S)-2-oxo(5,5-²H₂)pyrrolidin-3-yl]ethyl}-4-(trifluoromethyl)-L-prolinamide.

23. A compound of claim 1 having the Formula

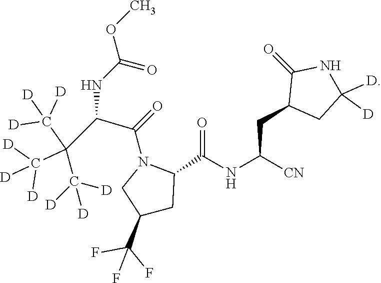

24. A compound of claim 1 having the Formula

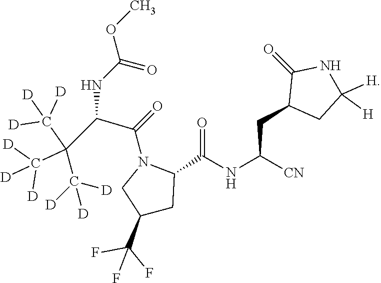

25. A compound of claim 1 having the Formula

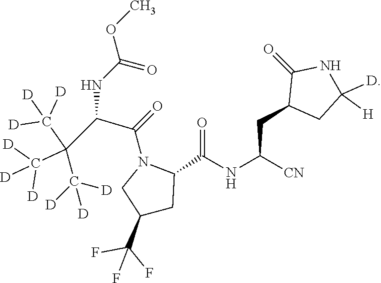

26. A pharmaceutical composition comprising a compound of any one of claims 1 to 25 and at least one pharmaceutically acceptable excipient.

27. A method for treating a coronavirus infection or coronavirus infection related disorder, comprising administering to a subject in need thereof a therapeutically effective amount of a compound of any one of claims 1 to 25.

28. A compound of any one of claims 1 to 25, for use as a medicament.

29. A compound of any one of claims 1 to 25, for use in the treatment of a coronavirus infection or coronavirus infection related disorder.
